(19) United States
(12) Patent Application Publication
(10) Pub. No.: US 2011/0318360 A1
(43) Pub. Date: Dec. 29, 2011

(54) ANTI-LIPID ANTIBODIES

(75) Inventors: Barton F. Haynes, Durham, NC (US); M. Anthony Moody, Durham, NC (US); Bua-Xin Liao, Durham, NC (US)

(73) Assignee: DUKE UNIVERSITY, Durham, NC (US)

(21) Appl. No.: 12/737,987

(22) PCT Filed: Sep. 8, 2009
(86) PCT No.: PCT/US2009/005023

§ 371 Date: Mar. 7, 2011

Related U.S. Application Data
(60) Provisional application No. 61/136,449, filed on Sep. 5, 2008.
(60) Provisional application No. 61/136,884, filed on Oct. 10, 2008.

(51) Int. Cl. A61K 39/395; A61P 31/18

(52) U.S. Cl. 424/154.1

(57) ABSTRACT

The present invention relates, in general, to anti-lipid antibodies and, in particular, to methods of inhibiting HIV-I infection using anti-lipid (e.g. anti-phospholipid) antibodies.

This application claims priority from U.S. Provisional Appln. No. 61/136,449, filed Sep. 5, 2008, and U.S. Provisional Appln. No. 61/136,884, filed Oct. 10, 2008, the entire contents of which are hereby incorporated by reference

This invention was made with government support under Grant No. U01 AI 067854, awarded by the National Institutes of Health. The government has certain rights in the invention.

### TECHNICAL FIELD

The present invention relates, in general, to anti-lipid antibodies and, in particular, to methods of inhibiting HIV-1 infection using anti-lipid (e.g. anti-phospholipid) antibodies.

### BACKGROUND

The development of strategies to utilize human antibodies that potently inhibit HIV-1 infection of T cells and mononuclear phagocytes is a high priority for treatment and prevention of HIV-1 infection (Mascola et al, J. Virol. 79:10103-10107 (2005)). A few rare human monoclonal antibodies (mAbs) against gp160 have been isolated that can broadly neutralize HIV-1 in vitro, and can protect non-human primates from SHIV infections in vivo (Mascola et al, Nat. Med. 6:207-210 (2000), Baba et al, Nat. Med. 6:200-206 (2000)). These mAbs include antibodies 2F5 and 4E10 against the membrane proximal region of gp41 (Muster et al, J. Virol. 67:6642-6647 (1993); Stiegler et al, AIDS Res. & Hum. Retro. 17:1757-1765 (2001), Zwick et al, J. Virol. 75:10892-10905 (2001)), IgG1b12 against the CD4 binding site of gp120 (Roben et al, J. Virol. 68:4821-4828 (1994)), and mAb 2G12 against gp120 high mannose residues (Sanders et al, J. Virol. 76:7293-7305 (2002)).

HIV-1 has evolved a number of effective strategies for evasion from neutralizing antibodies, including glycan shielding of neutralizing epitopes (Wei et al, Nature 422:307-312 (2003)), entropic barriers to neutralizing antibody binding (Kwong et al, Nature 420:678-682 (2002)), and masking or diversion of antibody responses by non-neutralizing antibodies (Alam et al, J. Virol. 82:115-125 (2008)). Despite intense investigation, it remains a conundrum why broadly neutralizing antibodies against either the gp120 CD4 binding site or the membrane proximal region of gp41 are not routinely induced in either animals or man.

One clue as to why broadly neutralizing antibodies are difficult to induce may be found in the fact that all of the above-referenced mAbs have unusual properties. The mAb 2G12 is against carbohydrates that are synthesized and modified by host glycosyltransferases and are, therefore, likely recognized as self carbohydrates (Calarese et al, Proc. Natl. Acad. Sci. USA 102:13372-13377 (2005)). 2G12 is also a unique antibody with Fabs that assemble into an interlocked VH domain-swapped dimers (Calarese et al, Science 300:2065-2071 (2003)). 2F5 and 4E10 both have long CDR3 loops, and react with multiple host antigens including host lipids (Zwick et al, J. Virol. 75:10892-10905 (2001), Alam et al, J. Immun. 178:4424-4435 (2007), Zwick et al, J. Virol. 78:3155-3161 (2004), Sun et al, Immunity 28:52-63 (2008)). Similarly, IgG1b12 also has a long CDR3 loop and reacts with dsDNA (Haynes et al, Science 308:1906-1908 (2005), Saphire et al, Science 293:1155-1159 (2001)). These findings, coupled with the perceived rarity of clinical HIV-1 infection in patients with autoimmune disease (Palacios and Santos, Inter. J. STD AIDS 15:277-278 (2004)), have prompted the hypothesis that some species of broadly reactive neutralizing antibodies are not made due to downregulation by immune tolerance mechanisms (Haynes et al, Science 308:1906-1908 (2005), Haynes et al, Hum. Antibodies 14:59-67 (2005)). A corollary of this hypothesis is that some patients with autoimmune diseases may be “exposed and uninfected” subjects with some type of neutralizing antibody as a correlate of protection (Kay, Ann. Inter. Med. 111:158-167 (1989)).

Key to evaluation of this hypothesis is the identification of human antibodies from autoimmune disease patients that inhibit HIV-1 infectivity. The present invention results, at least in part, from the demonstration that human monoclonal anti-lipid antibodies can be isolated from patients with autoimmune diseases, such as primary anti-phospholipid antibody syndrome (APAS) and systemic lupus erythematosus, as well as from PBL antibody libraries from healthy subjects, and that such antibodies can inhibit HIV-1 infectivity in peripheral blood mononuclear cells (PBMC) in vitro. HIV-1 inhibiting anti-lipid antibodies do not require β2-glycoprotein-1 to bind to lipids, and can be effective up to 48 hours after HIV-1 contact with target T cells. Such antibodies broadly neutralize transmitted CCR5-utilizing, but not CXCR4-utilizing, HIV-1 strains by binding to PB monocytes, and likely other antigen-presenting cells, and inducing the CCR5-binding chemokines, MIP-1α and MIP1-β. That this class of antibodies is able to inhibit HIV-1 infectivity of peripheral blood mononuclear cells (PBMCs) 48 hours after addition of HIV-1 to PBMC cultures and acts on only R5 viruses, demonstrates the utility of these antibodies as therapeutic agents in the setting of either prevention of transmission of HIV-1 or in the setting of post-exposure prophylaxis.

### SUMMARY OF THE INVENTION

The present invention relates generally to anti-lipid antibodies. More specifically, the invention relates to a method of inhibiting HIV-1 infection of T-cells using anti-lipid (e.g., anti-phospholipid) antibodies.

Objects and advantages of the present invention will be clear from the description that follows.

### BRIEF DESCRIPTION OF THE DRAWINGS

FIG. 1. The anti-lipid mAbs that inhibit HIV-1 infectivity are not dependent on binding to β-2-glycoprotein 1 for their lipid binding activity, whereas the anti-lipid mAbs that do not inhibit HIV-1 infectivity are dependent on binding to β-2-glycoprotein 1 for their lipid binding activity. (Left to right: IS4, CL1, P1, 11.31 (PGN 632), bavituximab, B1, B2, 1N11 (PGN 635), 3J05 (PGN 634), negative ctl.)

FIG. 2. Lack of virus capture by anti-lipid mAbs. A panel of mAbs was captured and incubated with HIV-1 BG1168 virions; virus capture was measured by ELISA of released p24. Only mAbs 7B2 against the gp41 immunodominant region and F39F against the gp120 V3 loop captured virions in the absence of soluble CD4 triggering. Anti-gp120 CCR5 binding site mAb 17b was able to capture in the presence of CD4 triggering but not without it. In contrast, none of the anti-lipid mAbs captured virions in this assay. Similar results were seen for HIV-1 isolate SF162. (Antibody P3 is mouse myeloma, P3×63-Ag8, ATCC Number CRL-1580 or CRL-1579; antibody A32 is human anti-HIV-1 envelope from James Robinson/Tulane University.)

FIG. 3. Anti-lipid mAbs inhibit SHIV SF162P3 or QH0692 by binding to host cells. Antibodies IS4 and CL1 tested against B.QH0692 and P1 and 11.31 (PGN 632) tested against SHIV SF162P3 were added to the PBMC assay either by preincubating the mAb with the virus stock for one hour prior to the addition of cells (open point curves) or by incubating the target cells with antibody for one hour followed by washing excess antibody and then addition of virus stock (closed point curves). For each of the mAbs tested, no significant change in inhibitory activity was seen suggesting that this activity was primarily directed against the target cells.

FIGS. 4A and 4B. FIG. 4A. Binding of anti-lipid mAbs to phytohemaglutinin (PHA)-activated human PBMC. MAbs were incubated with PBMC and then with goat anti-human Ig conjugated to FITC, and assayed in indirect immunofluoresence by flow cytometry. FIG. 4B. HIV-1 MN infected H9 human T cells bound anti-gp120 V3 mab F39F indicating productive HIV-1 infection. Mabs 2F5, 4E10, bavituximab, and 11.31 (PGN 632) bound the surface of viable infected cells with similar potency. Limited binding of viable cells was demonstrated by mAb P1, and this correlated with the P1 mAb being the least potent of those anti-lipid antibodies that could inhibit HIV-1 infectivity.

FIG. 5. Binding of 11.31 (PGN 632) to the surface of PHA-activated PBMC. Shown are PBMC labeled with cholera toxin B (CTB) for surface staining of lipids, and with 11.31 (PGN 632) labeled with phycoerythrin. Colocalization of both 11.31 (PGN 632) mAb and CTB to the membrane of the PBMC cells is shown (arrows).

FIG. 6. Blocking of inhibition of HIV-1 B.6535 by preincubation with various polymorphic forms of lipids. The mAbs preincubated with PBS, 0.5 mM DOPE (in hexagonal II polymorphic form), or 0.5 mM CL (in liposome form) show varying effects. MAbs P1 and IS4 demonstrated little (or minimal) change in potency with lipid incubation. CL1 and 11.31 (PGN 632) showed no effect when reacted with DOPE but were potently inhibited by CL with reduction of 11-fold and 200-fold, respectively. P1 showed modest inhibition with DOPE incubation and modest enhancement with CL incubation. Thus, the correct polymorphic form of a lipid was found for CL1 and 11.31 (PGN 632) mAbs. These data suggest differences in reactivity of CL1 and 11.31 (PGN 632) vs. IS4 and P1.

FIGS. 7A and 7B. PBMC were isolated by density gradient centrifugation from discarded white cell buffy coats obtained from the American Red Cross (Carolinas Blood Services Region) under an IRB-approved protocol. Cells were either used as prepared or were further purified using an autoMACS Pro separator (Miltenyi Biotec, Auburn, Calif.). The resulting cells were checked for purity by post-purification FACS analysis. Monocytes (94% pure, <1% residual T-cells), monocyte depleted PBMC (<1% residual monocytes), CD4+ T-cells (93% pure, <0.5% CD8+ T-cells, <0.5% monocytes), CD4+ T-cell depleted PBMC (<1% residual CD4+ T-cells), and unpurified PBMC were infected with HIV-1 B.PVO in the presence or absence of serial dilutions of monoclonal antibodies. FIG. 7A. Antibody neutralization determined as the concentration neutralizing 80% of infection seen in control wells. Antibody 11.31 (PGN 632) neutralized only in those cell samples that contained monocytes and showed no inhibition of infection in samples depleted of monocytes. FIG. 7B. Antibody neutralization determined as a reduction of infection compared to control wells without antibody. Antibodies 11.31 (PGN 632) and CL1 reduced the infection of monocytes by 98% and 93% respectively; neither antibody inhibited the infection of purified CD4+ T-cells. In contrast, 2G12 and IgG1b12 inhibited the infection of both cell types, with 2G12 being the more potent antibody.

FIG. 8. Purified monocytes or CD4+ T-cells were pretreated with 11.31 for 30 minutes at 37 C and then washed. The pretreated cells were then added to cultures of CD4+ T-cells and infected with HIV-1 B.6535 and inhibition was measured as a reduction of p24 production compared to untreated control wells. Pretreatment of monocytes resulted in an 87% reduction of infection while pretreatment of CD4+ T-cells resulted in only 35% reduction.

FIGS. 9A-9C. Anti-lipid antibodies induce R5 chemokines from PBMC and can, in the presence of HIV-1, combine to induce high levels of CCR5 chemokines from PB monocytes. FIG. 9A shows that anti-lipid antibodies CL1 and 11.31 (PGN 632) induce chemokines in the absence of HIV-1 and induce higher levels of chemokines in the presence of HIV-1. FIG. 9B shows a summary of the same data of CL1 only with data taken from the 24 hour timepoint. FIG. 9C shows a second experiment using a different individual's PBMC—in this case, the lipid antibody alone induced maximal levels of chemokines from PBMC at 24 hours. In both, the lipid antibody effect was most pronounced on MIP-1α and MIP-1β and not on RANTES. In addition, there was no effect on the CXCR4 chemokine SDF-1, explaining the effect of the lipid antibodies on R5HIV-1 isolates exclusively.

FIG. 10. Blocking of HIV-1 inhibition activity of anti-lip antibody by anti-chemokine antibodies. PBMC were first activated with 5 μg/ml PHA at 37° C. overnight and incubated with no antibody or with MAb CL 1 at sub-saturating concentration (3.3 μg/ml) in the presence of the indicated (in the x axis) neutralizing anti-chemokine MAbs or control (P3) at 8.3 μg/ml. Culture supernatants were harvested 5 days after HIV-1 infection and assayed for p24 production using the p24 assay kit (PerkinElmer).

FIGS. 11A and 11B. FIG. 11A. Sequences for mAb 11.31 (PGN 632). FIG. 11B. Sequences for CL1.

FIGS. 12A and 12B. FIG. 12A. Anti-lipid antibodies that inhibit HIV-1 infectivity in the PBMC assay are non-pathogenic and do not depend on β-2-glycporotein-1 for binding to lipids. This observation is important because, in general, those antibodies that do not require β-2-glycporotein-1 for binding to lipids are non-pathogenic, whereas pathogenic anti-lipid antibodies do require β-2-glycporotein-1 for antibody binding to lipids (DeGroot et al, J. Thromb. Haemost. 3:1854-1860 (2005)). Of the antibodies studied, only those that did not depend on β-2-glycporotein-1 for binding to lipids were able to inhibit HIV-1 infectivity. All of the other antibodies that do depend on β-2-glycoprotein-1 for binding to lipids do not have the ability to inhibit HIV-1 infectivity. Thus, these non-β-2-glycoprotein-1-dependent antibodies are in a class of non-pathogenic anti-lipid antibodies with “natural” anti-lipid antibodies that are commonly made to lipids by humans (Alving, Biochem. Soc. Trans. 12:342-344 (1984)), and are frequently seen following infection with a variety of infectious agents, including syphilis and HIV-1 (so-called “infectious lipid or cardiolipin antibodies” (Asherson et al, Ann. Rheum. Dis. 62:388-393 (2003) Silverstri et al, Blood 87:5185-5195 (1996))). FIG. 12B. Anti-lipid antibodies inhibit R5HIV-1 primary isolates with greater breadth than 2F5, 2G12 and 1b12 mAbs. Whereas all the CCR5-utilizing HIV strains are neutralized in unprecedented breadth and potency by the four non-pathogenic lipid mAbs IS4, CL1, P1 and 11.31 (PGN 632), none of the CXCR4-utilizing HIV or SHIV strains are prevented from infecting PBMC by the lipid antibodies. This striking effect of the anti-lipid antibodies, coupled with the observations that the anti-lipid antibodies acted only on host cells and not the virus (see FIGS. 2 and 3), strongly suggested that the lipid antibodies were inducing a factor specific from host cells for inhibiting CCR5-utilizing viruses. This is significant since nearly all of the viruses that traverse the mucosal bottleneck of mucosal transmission are CCR5-utilizing viruses (Keele et al, Proc. Natl. Acad. Sci. 105:7557-7, Epub 2008 May 19 (2008)).

FIG. 13. Cardiolipin competitively inhibits the ability of mAbs 11.31 (PGN 632) and CL1 is block HIV-1 infection in PBMCs.

FIGS. 14A-14D. Activity of human antibodies for 75 donor PBMC. FIG. 14A. 11.31. FIG. 14B. IgG1b12. FIG. 14C. 4E10. FIG. 14D. HIVIG.

FIGS. 15A-15F. Incubation of monocytes with anti-lipid monoclonal antibodies stimulates polykaryon formation. FIG. 15A. 11.31. FIG. 15B. CL1. FIG. 15C. IS4. FIG. 15D. P1. FIG. 15E. LPS. FIG. 15F. 17b.

FIGS. 16A and 16B. Inhibition of HIV-1 WITO pseudovirus in TZM-bl cells by culture supernatant from PBMC incubated with anti-lipid antibodies. FIG. 16A. 11.31. FIG. 16B. CL1.

FIG. 17. Anti-lipid human monoclonal antibodies inhibit HIV-1 infection of PBMC by binding to host cells.

### DETAILED DESCRIPTION OF THE INVENTION

The present invention relates, in one embodiment, to a method of inhibiting infection of cells (e.g. T-cells) of a subject by a CCR5-tropic strain of HIV-1. The method comprises administering to the subject (e.g., a human subject) an anti-human cell antibody (for example, an anti-lipid (e.g., anti-phospholipid) antibody), such as mAb 11.31 (PGN 632) or CL 1), or fragment thereof, in an amount and under conditions such that the antibody, or fragment thereof, binds to cells of the patient that: i) can produce CCR5-binding chemokines, and ii) have on their surface an antigen recognized by the antibody. Binding of the antibody, or fragment thereof, induces the production of the CCR5-binding chemokines by the cells, either in the absence or in the presence of the CCR5-tropic strain of HIV-1, to a level sufficient to inhibit infection of HIV-1 susceptible cells that utilize the CCR5-receptor (e.g., T-cells). Advantageously, the antibody, or fragment thereof, is administered within 48 hours of exposure of the subject to the CCR5-tropic strain of HIV-1.

Anti-lipid antibodies suitable for use in the invention can be derived from healthy control subjects and from patients with primary and secondary forms of APAS (e.g., from antibody libraries generated from peripheral blood lymphocytes (PBLs) from such patients). Examples of such antibodies from SLE patients (CL1, P1), from an anti-phospholipid syndrome patient (IS4) and from a normal subject (11.31 (PGN 632)) are found in Table 3. In addition, HIV-1 itself stimulates the production of these types of antibodies after HIV-1 infection (see data with ACL4 mAb derived from a subject 3 months after HIV-1 transmission in Table 1).

TABLE 1
Inhibition of HIV-1 by anti-Lipid antibodies In PBMC-based
neutralization assays Using LucR-incorporated HIV-1.

| | HIV-1 Isolates | | |
|---|---|---|---|
| | HIV-WITO. | HIV-WITO. | HIV-WEAU3-3. |
| | LucR.T2A. | LucR.T2A. | LucR.T2A. |
| Antibody | ecto/hPBMC* | ecto/hPBMC* | ecto/hPBMC# |
| IS4 | 0.08 | <0.02 | >50.00 |
| P1 | <0.02 | <0.02 | >50.00 |
| 11.31 | <0.02 | <0.02 | >50.00 |
| A32 | >50.00 | >50.00 | >50.00 |
| 4E10 | 0.09 | 0.16 | 22.24 |
| ACL4 | 1.00 | 1.33 | >50.00 |
| CL1 | <0.02 | <0.02 | >50.00 |
| Synagis | >50.00 | >50.00 | >50.00 |
| 2F5 | 0.97 | 4.22 | 6.44 |
| 4E10 | 0.05 | 0.33 | 4.52 |

*CCR5 HIV-1 isolate;
#CXCR4 isolate.

Antibodies derived from patients and healthy subjects as described above can be further matured to optimize for high affinity lipid (e.g., phospholipid) binding. Preferred antibodies bind directly to phospholipids (e.g., phosphatidylserine (PS)) on the surface of cells (e.g., monocytes) that produce CCR5-binding chemokines, that is, they do not require β2-glycoprotein-1 to bind. Binding to domain I of β2-glycoprotein-1 has been associated with pathogenicity of anti-phospholipid antibodies found in APAS and other autoimmune syndromes (DeGroot et al, J. Thromb. Haemost. 3:1854-1860 (2005)). Anti-lipid antibodies suitable for use in the invention can broadly neutralize CCR5— but not CXCR4-utilizing HIV-1 strains. Preferred therapeutic antibodies of the invention do not require β-2-glycoprotein-1 in order to bind lipids. Such antibodies can arise in and be derived from subjects that do not have complications of thrombosis resulting from the isolated antibody (ACL4 being an example of such an antibody).

In accordance with the invention, the anti-lipid antibodies can be administered prior to contact of the subject or the subject's immune system/cells with CCR5-utilizing HIV-1 or within about 48 hours of such contact. Administration within this time frame can maximize inhibition of infection of vulnerable cells of the subject (e.g., T-cells) with CCR5-tropic HIV-1. This mode of inhibition of HIV-1 is particularly effective for modifying or inhibiting the transmission event, since virtually all of the transmitted HIV-1 viral quasispecies are CCR5-tropic (Keele et al, Proc. Natl. Acad. Sci. 105:7552-7557, Epub 2008 May 19 (2008)).

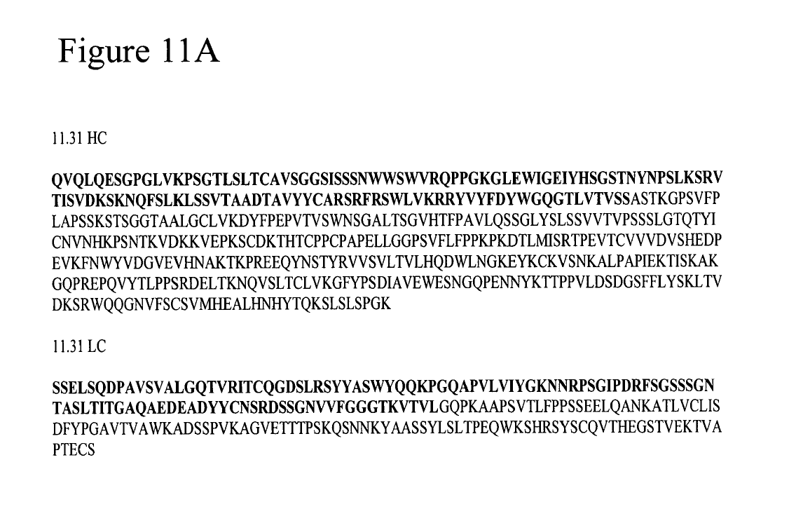

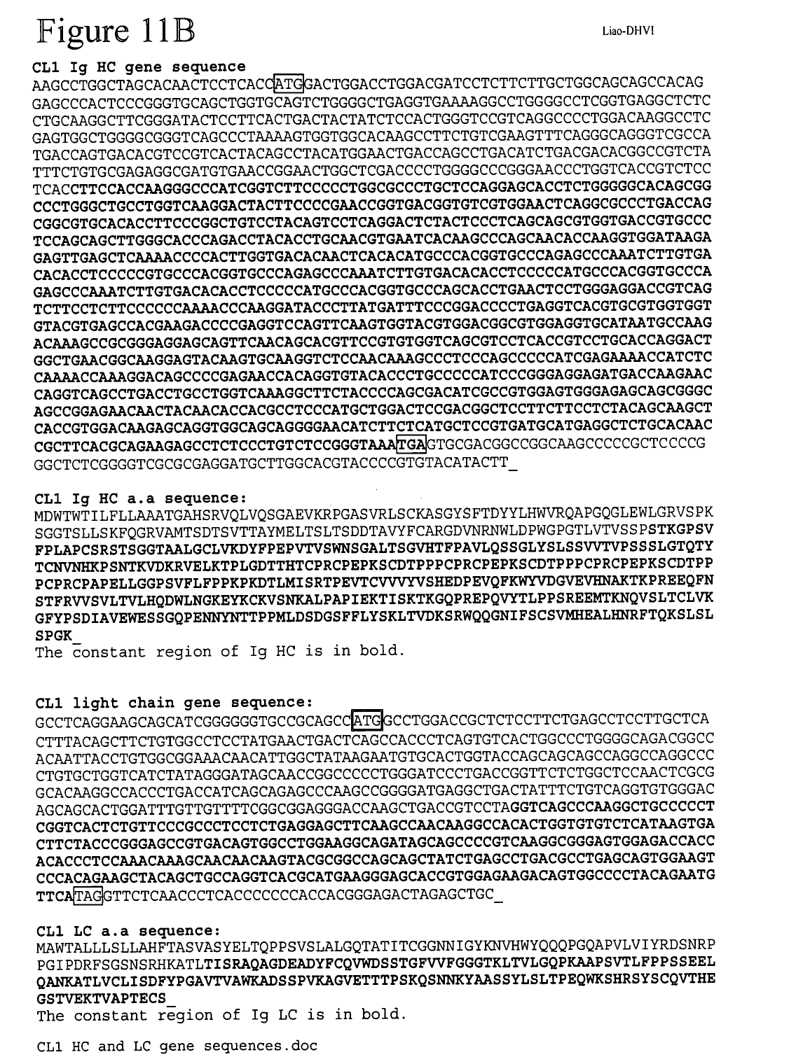

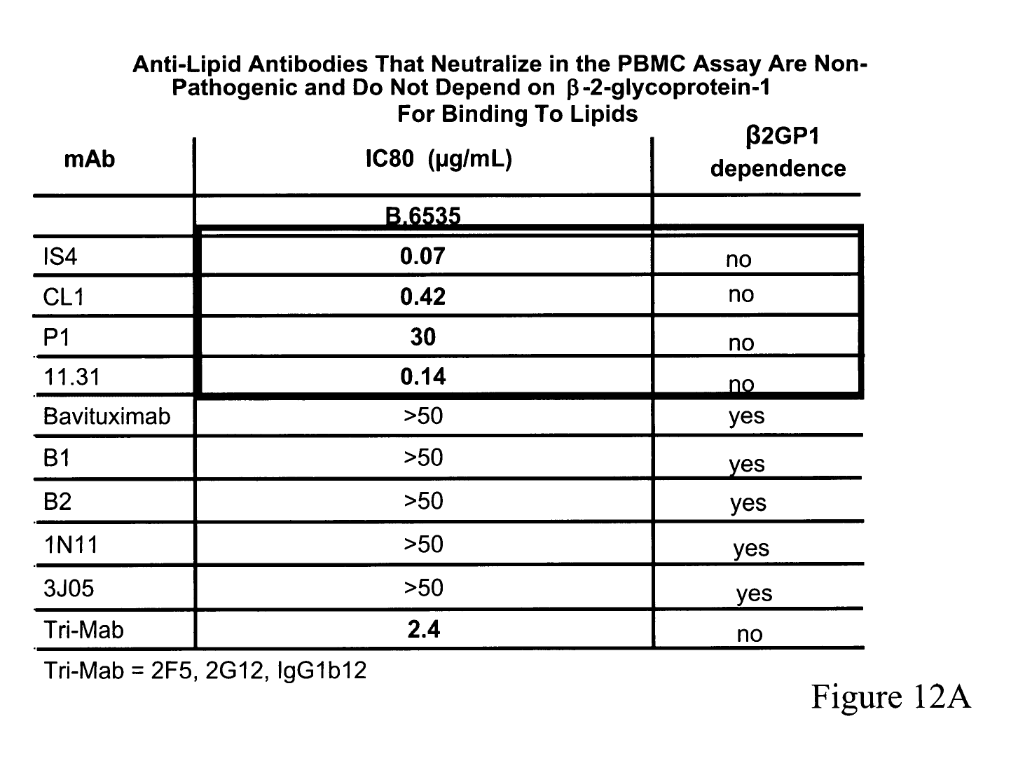

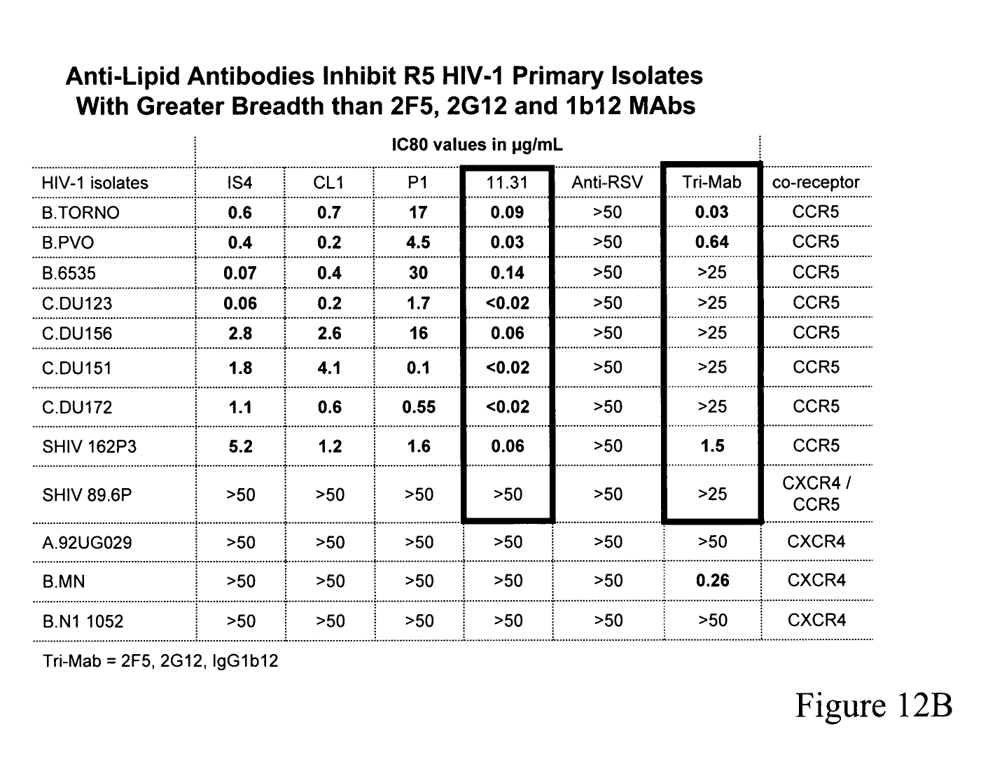

One preferred antibody for use in the invention is mAb 11.31 (PGN 632). This antibody was derived from an antibody library generated from PBLs of healthy donors. Whether it reflects an antibody that was being made at the time of production of the antibody library is not known. The original antibody isotype was IgM or IgD that was then converted to IgG and was further matured to optimize for high affinity PS binding. The potency of mAb 11.31 (PGN 632) for inhibition of CCR5-utilizing HIV-1 infection of PBMCs is broader than any other antibody reported. The sequences of the variable domains for 11.31 (PGN 632) are set forth in Table 2 (the IgG sequences are shown in FIG. 11A).

TABLE 2

| SEQ | | |
|---|---|---|
| ID NO | Description | Sequence |
| 1 | VH domain | caggtgcagctgcaggagtcgggcccaggactggtgaagccttcggggac |
| | (nt) | cctgtccctcacctgcgctgtctctggtggctccatcagcagtagtaact |
| | | ggtggagttgggtccgccagcccccagggaaggggctggagtggattggg |
| | | gaaatctatcatagtgggagcaccaactacaacccgtccctcaagagtcg |
| | | agtcaccatatcagtagacaagtccaagaaccagttctccctgaagctga |
| | | gctctgtgaccgccgcggacacggccgtgtattactgtgcgaggagtcgt |
| | | tttaggtcgtggctggtaaagcgccggtatgtctactttgactactgggg |
| | | ccagggaaccctggtcaccgtctcctca |
| 2 | VL domain | tcctctgagctgagtcaggaccctgctgtgtctgtggccttgggacaga |
| | (nt) | cagtcaggatcacatgccaaggagacagcctcagaagctattatgcaagc |
| | | tggtaccagcagaagccaggacaggcccctgtacttgtcatctatggtaa |
| | | aaacaaccggccctcagggatcccagaccgattctctggctccagctcag |
| | | gaaacacagcttccttgaccatcactggggctcaggcggaagatgaggct |
| | | gactattactgtaactcccgggacagcagtggtaacgtggtattcggcgg |
| | | agggaccaaggtgaccgtccta |
| 3 | VH domain | QVQLQESGPGLVKPSGTLSLTCAVSGGSISSSNWWSWVRQPPGKGLEWIG |
| | (aa) | EIYHSGSTNYNPSLKSRVTISVDKSKNQFSLKLSSVTAADTAVYYCARSR |
| | | FRSWLVKRRYVYFDYWGQGTLVTVSS |
| 4 | VL domain | SSELSQDPAVSVALGQTVRITCQGDSLRSYYASWYQQKPGQAPVLVIYGK |
| | (aa) | NNRPSGIPDRFSGSSSGNTASLTITGAQAEDEADYYCNSRDSSGNVVFGG |
| | | GTKVTVL |

Also preferred for use in the method of the invention is CL1. The sequences of the heavy and light chain genes are shown in FIG. 11B, with the amino acid sequences. Other antibodies derived from healthy individuals, HIV-1 infected subjects (such as ACL4), subjects infected with other agents (such as syphilis) or autoimmune disease patients, or fragments of such antibodies, can also be used in the instant method.

As indicated above, either the intact antibody or fragment (e.g., antigen binding fragment) thereof can be used in the method of the present invention. Exemplary functional fragments (regions) include scFv, Fv, Fab′, Fab and F(ab′)₂ fragments. Single chain antibodies can also be used. Techniques for preparing suitable fragments and single chain antibodies are well known in the art. (See, for example, U.S. Pat. Nos. 5,855,866; 5,877,289; 5,965,132; 6,093,399; 6,261,535; 6,004,555; 7,417,125 and 7,078,491 and WO 98/45331.) The invention also includes variants of the antibodies (and fragments) disclosed herein, including variants that retain the binding properties of the antibodies (and fragments) specifically disclosed, and methods of using same in the present method.

The antibodies, and fragments thereof, described above can be formulated as a composition (e.g., a pharmaceutical composition). Suitable compositions can comprise the anti-lipid antibody (or antibody fragment) dissolved or dispersed in a pharmaceutically acceptable carrier (e.g., an aqueous medium). The compositions can be sterile and can in an injectable form. The antibodies (and fragments thereof) can also be formulated as a composition appropriate for topical administration to the skin or mucosa. Such compositions can take the form of liquids, ointments, creams, gels and pastes. Standard formulation techniques can be used in preparing suitable compositions. The antibodies can be formulated so as to be administered as a post-coital douche or with a condom.

While many the anti-lipid antibodies suitable for use in the present method have been identified by virtue of their reactivity with cardiolipin (CL), CL is not expressed on the cell surface of viable, activated or apoptotic cells, but rather is a lipid of mitochondrial membranes. All four of the mAbs shown in the Example below to inhibit HIV-1 infectivity, while binding to CL, also bind to PS. The data provided in the Example indicate that PS is one of the relevant cell surface target cell molecule.

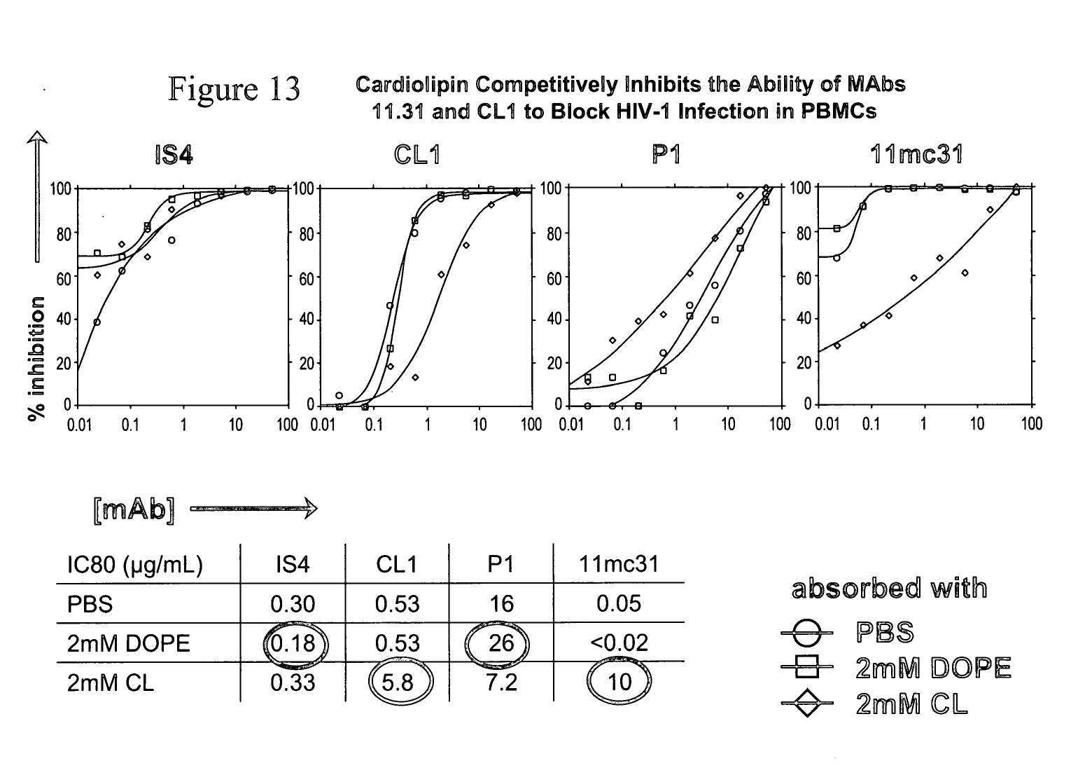

That anti-lipid antibodies only inhibit the infectivity of CCR5-utilizing primary isolates has significance for the mechanism of inhibition of infectivity and for the setting of utility of anti-lipid antibodies in inhibiting HIV-1 infection. That select anti-lipid antibodies (e.g. CL1 and 11.31 (PGN 632)) can inhibit HIV-1 infection up to 48 hours after addition of the virus show that they do not block virion binding and attachment. The data provided in the Example are compatible with the mode of action of the mAbs being induction of chemokines from monocytes and other chemokine producing cells. (See FIG. 13.) That the anti-lipid antibodies act up to 48 hours after infection show their utility for prophylaxis in, for example, the following settings:

i) in the setting of anticipated known exposure to HIV-1 infection, the anti-lipid antibodies described herein (or binding fragments thereof) and be administered prophylactically (e.g., IV or topically) as a microbiocide,

ii) in the setting of known or suspected exposure, such as occurs in the setting of rape victims, or commercial sex workers, or in any heterosexual transmission with out condom protection, the anti-lipid antibodies described herein (or fragments thereof) can be administered as post-exposure prophylaxis, e.g., IV or topically, and

iii) in the setting of Acute HIV infection (AHI) with an CCR5 transmitted virus, the anti-lipid antibodies described herein (or binding fragments thereof) can be administered as a treatment for AHI to control the initial viral load and preserve the CD4+ T cell pool and prevent CD4+ T cell destruction.

Suitable dose ranges can depend on the antibody and on the nature of the formulation and route of administration. Optimum doses can be determined by one skilled in the art without undue experimentation. Doses of antibodies in the range of 10 ng to 20 μg/ml can be suitable (both administered and induced).

Certain aspects of the invention can be described in greater detail in the non-limiting Examples that follows. (See also U.S. Provisional Application No. 61/136,449, the entire content of which is incorporated herein by reference.)

### Example 1

### Experimental Details

Antibodies. MAbs used in this study and their characteristics are shown in Table 3. IS4 is a human mAb derived from a patient with primary anti-phospholipid antibody syndrome (APAS) (Zhu et al, J. Haematol. 105:102-109 (1999)) (see accession numbers AF417845 and AF417851). CL1, P1, B1, and B2 are human mAbs derived from a patient with secondary APAS and systemic lupus erythematosus (SLE) (Wei-Shiang et al, Arth. Rheum. 56:1638-1647 (2007)). MAbs 11.31 (PGN 632), J305 (PGN 634), and 1N11 (PGN 635) are recombinant mAbs derived from an antibody library generated from blood of healthy subjects and engineered for optimal binding to PS. Each cell line was grown in serum-free media and whole immunoglobulin was purified using protein A/G preparative columns. Synagis™ (palivizumab) is a humanized mAb against the F-protein of respiratory syncytial virus and was purchased from MedImmune, Inc. (Gaithersburg, Md.). Anti-gp41 membrane proximal external region (MPER) mAbs 2F5 and 4E10 were purchased from Polymun Scientific (Vienna, Austria). MAbs 7B2, F39F, 17b, and A32 were generous donations of James Robinson (Tulane University, New Orleans, La.). Goat anti-human IgG (H+L) was purchased from KPL, Inc (Gaithersburg, Md.) and titered to determine optimal concentration. β2-glycoprotein-1 Fc dimer is a dimeric form of the full length (domains I-V) of β2-glycoprotein-1 spliced to an IgG1 Fc (Peregrine Pharmaceuticals, Tustin, Calif.).

TABLE 3
Anti-lipid antibodies in this study.

| | binds | binds | binds directly | binding to CL/PS | |
|---|---|---|---|---|---|
| Mab | CL/PS | HIV-1 Env | to β2GP1 | dependent on β2GP1 | Mab origin |
| IS4 | + | − | + | no | APAS subject |
| CL1 | + | − | ++ | no | SLE subject |
| P1 | + | − | +++ | no | SLE subject |
| 11.31 | + | − | +/− | no | uninfected and healthy |
| (PGN 632) | | | | | subject * |
| Bavituximab | + | − | +++ | yes | humanized mouse Mab |
| B1 | + | − | +++ | yes | SLE subject |
| B2 | + | − | + | yes | SLE subject |
| 1N11 | + | − | +++ | yes | uninfected and healthy |
| (PGN 635) | | | | | subject * |
| 3J05 | + | − | + | yes | uninfected and healthy |
| (PGN 634) | | | | | subject * |
| 2F5 | + | + | + | no | HIV-1 + subject |
| 4E10 | + | + | + | no | HIV-1 + subject |

* Derived from antibody libraries from healthy subjects modified for improved bindind to PS.

Recombinant Envs and Other Reagents. PBS and PBS with 1% BSA were purchased from Gibco Invitrogen (Grand Island, N.Y.). Methanol-free formaldehyde 10% was purchased from Polysciences, Inc, (Warrington, Pa.). Recombinant gp140 CF or CFI group M consensus CON-S, JRFL, and X Env oligomers were produced in recombinant vaccinia viruses as secreted proteins as described (Liao et al, Virology 553:268-282 (2006)).

Patient and control specimens. Healthy control subjects and patient samples were acquired under clinical protocols approved by the Duke University IRB. Patient samples 1-10 were obtained from a repository of antiphospholipid antibody syndrome (APAS) patient samples maintained at Duke University Medical Center. Patient samples 11-30 came from a selection of subjects recruited under the CHAVI 005 protocol designed to recruit patients with autoimmune disease and healthy controls. All samples were tested for the presence of anti-cardiolipin antibodies and were screened by a standard HIV-1 ELISA. The CHAVI 005 samples were also tested by RNA PCR for viral load. All samples tested were negative for anti-HIV antibodies and had no detectable HIV-1 viral RNA.

Isolation of human CD4+ T cells and CD14+ monocytes. PBMC obtained as discarded buffy coats from the American Red Cross or from leukapheresis of uninfected normal subjects were enriched for CD4+ T cells using an autoMACS™ Pro Separator (Milteny Biotech, Auburn, Calif.) using negative selection or were enriched for monocytes using an elutriator. Resulting cell preparations were analyzed by staining with CD3, CD4, and CD8 antibodies and analysis on either a BD LSR II (BD Biosciences, Mountain View, Calif.) or a Guava EasyCyte Mini-SSC system (Guava Technologies, Hayward, Calif.). All preparations were ≧95% CD3+ CD4+ or >95% CD14+.

Surface plasmon resonance and flow cytometry. Binding of mAbs to substrates were studied using surface plasmon resonance (SPR) and flow cytometry. SPR studies were performed using standard techniques on a BIAcore 3000 (BIAcore, Inc, Piscataway, N.J.). Flow cytometric studies were performed on the human T cell line H9 (ATCC, Manassas, Va.) on human peripheral blood mononuclear cells (PBMC) or on blood monocytes. Staining for flow cytometry was performed at 37° C. with primary antibody incubated for 30-60 min and secondary for 30 min. Flow samples were fixed in 1-2% methanol-free formaldehyde in PBS and stored at 4° C. prior to analysis on a BD LSR II flow cytometer (BD Biosciences, San Jose, Calif.).

Neutralization assay in TZM-bl cells. Neutralizing antibody assays in TZM-bl cells were performed as described previously (Wei et al, Nature 422:307-312 (2003); Derdeyn et al, J. Virol. 74:8358-8367 (2000); Li et al, J. Virol. 79:10108-10125 (2005); Montefiori, D C pp 12.11.1-12.11.15, In Current Protocols in Immunology (2004)). Briefly, the adherent cells were disrupted by treatment with trypsin/EDTA before use. Patient sera were tested starting at 1:20 final dilution while mAbs were tested starting at 50 μg/mL final concentration. Both were titered using serial 3 fold dilutions. Pseudoviruses were added to the antibody dilutions at a predetermined titer to produce measurable infection and incubated for one hour. TZM-bl cells were added and incubated for 48 hours after which supernatant was measured by a luminometer. The data were calculated as a reduction in luminescence compared to control wells and reported as mAb IC50 in μg/ml (Montefiori, Current Protocols in Immunology, J. Coligan et al, eds., John Wiley & Sons, Inc., Hoboken, N.J. 12.11.11-12.11.15 (2004)).

Neutralization assay in PBMCs. PBMC assays were performed using whole virus preparations to infect PBMC with infection detected using p24 ELISA (Abbott, Chicago, Ill.). Mabs and human sera were incubated with virus or cells as noted and then free antibody washed away prior to infection (Pilgrim et al, J. Infect. Dis. 176:924-932 (1997)). Briefly, cryopreserved human PBMC were thawed and rested in culture for one day in IL-2 growth medium (RPMI 1640 with 2 mM L-glutamine, 25 mM HEPES, 20% heat-inactivated fetal bovine serum, 5% IL-2, 50 μg/mL gentamicin) containing phytohemagglutinin at 5 μg/mL. Cells were then washed and added to U-bottom wells containing antibody or serum dilutions as appropriate and incubated for one hour before adding HIV, SIV, or SHIV isolates at an appropriate dilution. After 24 hours the cells were washed four times with IL-2 growth medium and then incubated for a further 24 hours. Media (25 μL) was removed and incubated with 225 μL 0.5% Triton X-100 and then assayed by p24 ELISA. Data were calculated as a reduction of p24 production compared to control infected wells and expressed as mAb IC80 in μg/mL. Studies of mAbs preabsorbed with lipids were performed with antibody stocks incubated with 2 mM cardiolipin (CL), 2 mM dioleoylphosphatidylethanolamine (DOPE), or PBS at 37° C. for 2 h or overnight after which the mixture was assayed as above. Time course studies were performed by adding mAb at time 0, 24 h, 48 h, or 72 h. In these experiments, antibody was reintroduced after each wash step so that a constant concentration of antibody was present throughout the assay.

Antibody inhibition of HIV-1 induced syncytium formation. Syncytium inhibition assays were performed using 2,2′-dipyridyl disulfide (Aldrithiol™-2) inactivated virions supplied as a generous gift from Larry Arthur and Jeffrey Lifson (Frederick Research Cancer Facility, Frederick, Md.). Antibody prepared in serial dilution was incubated with inactivated virions for 1 h at 37° C. SUP-T1 cells, grown in 10% FBS in RPMI 1640 with 50 μg/mL gentamicin were added to the antibody-virus mixture and incubated for 16 h at 37° C., 5% CO₂. Syncytia were imaged using inverted phase-contrast microscopy and counted. Titers were expressed as the concentration of antibody that inhibited 90% of syncytium formation compared to wells containing no antibody.

Purification of IgG from human serum. IgG was purified from serum by affinity chromatography over Staph AG columns from Pierce Chemical Co.

Fluorescence microscopy of PBMC. PBMC were incubated with primary mAbs in the presence of aqua vital dye and AlexaFluor 555-labeled cholera toxin B (Invitrogen, Carlsbad, Calif.) for 30 min at 4° C. The samples were washed using 1% BSA in PBS and stained with goat-anti-human IgG (H+L)-FITC (KPL Inc, Gaithersburg, Md.) for 30 min. After final washing the cells were resuspended in minimal 1% BSA in PBS and maintained at 4° C. until viewed under fluorescence microscopy on an Olympus AX-70 microscope fitted with a SPOT CCD camera (Diagnostic Instruments, Sterling Heights, Mich.).

### Results

Screen of anti-lipid mAb ability of inhibit HIV-1 pseudovirus infection in single round infection assays and inhibit infectious virus in multiple round infection assays in PBMCs. The ability of the mAbs in Table 3 to inhibit the infection of HIV-1 Env pseudoviruses B.6535, B.PVO and C.DU123 was determined (Table 4A). None of the mAbs were found it inhibit any of the three pseudoviruses when cultured in the epithelial cell line TZMBL (an genital epithelial cell transfected with CCR5 and CD4). Next, a study was made of the ability of these antibodies to prevent the formation of syncytia induced by Aldrithiol™-2 inactivated virions in the SUP-T1 cell line (Table 4B). None of the anti-lipid antibodies prevented the formation of syncytia. The same mAb panel was then tested in a multiple round assay for the ability of mAbs to inhibit the infection of PBMCs by infectious HIV-1 primary isolates (Table 4C). In contrast to the lack of effect of anti-lipid mAbs in the pseudovirus and syncytium inhibition assays, it was found that four of the nine mAbs tested (11.31 (PGN 632), P1, IS4 and CL1) had potent neutralizing activity against B.PVO, B.6535, and C.DU123. Antibody 11.31 (PGN 632) was the most potent infection inhibitor, with IC80 against C.DU123 at <0.02 μg/ml.

TABLE 4A
Inability of anti-lipid antibodies to neutralize HIV-1 as enveloped
pseudoviruses in TZM-bl CD4+ CCR5+ epithelial cells

| | Mabs | | | | | | | | | |
|---|---|---|---|---|---|---|---|---|---|---|
| HIV-1 Env | IS4 | CL1 | P1 | 11.31 | bavituximab | B1 | B2 | 1N11 | 3J05 | 4E10 |
| pseudoviruses | (IC50 values in μg/mL) | | | | | | | | | |
| B.PVO | >50 | >50 | >50 | >50 | >50 | >50 | >50 | >50 | >50 | 2.2 |
| B.6535 | >50 | >50 | >50 | >50 | >50 | >50 | >50 | >50 | >50 | <2 |
| C.DU123 | >50 | >50 | >50 | >50 | >50 | >50 | >50 | >50 | >50 | <2 |

TABLE 4B
Inability of anti-lipid antibodies to inhibit fusion induced by Aldrithiol ™-2 inactivated
HIV-1 in the SUP-T1 T cell line

| | Mabs | | | | | | | | | | |
|---|---|---|---|---|---|---|---|---|---|---|---|
| HIV-1 | IS4 | CL1 | P1 | 11.31 | bavituximab | B1 | B2 | 1N11 | 3J05 | 2F5 | 4E10 |
| isolate | (IC90 values in μg/mL) | | | | | | | | | | |
| B.MN | >100 | >100 | >100 | >100 | >100 | >100 | >100 | >100 | >100 | <1.2 | <1.2 |
| B.ADA | >100 | >100 | >100 | >100 | >100 | >100 | >100 | >100 | >100 | <1.2 | <1.2 |
| B.AD8 | >100 | >100 | >100 | >100 | >100 | >100 | >100 | >100 | >100 | 3.4 | 10 |

TABLE 4C
Ability of anti-lipid antibodies to neutralize HIV-1
in a PBMC-based virus infection inhibition assay

| | Mabs | | | | | | | | | |
|---|---|---|---|---|---|---|---|---|---|---|
| HIV-1 | IS4 | CL1 | P1 | 11.31 | bavituximab | B1 | B2 | 1N11 | 3J05 | TriMab |
| isolates | (IC80 values in μg/mL) | | | | | | | | | |
| B.PVO | 0.43 | 0.21 | 4.5 | 0.03 | >50 | >50 | >50 | >50 | >50 | 0.64 |
| B.6535 | 0.07 | 0.42 | 30 | 0.14 | >50 | >50 | >50 | >50 | >50 | 2.4 |
| C.DU123 | 0.06 | 0.19 | <0.2 | <0.02 | 4.5 | >50 | >50 | 8.2 | >50 | >25 |

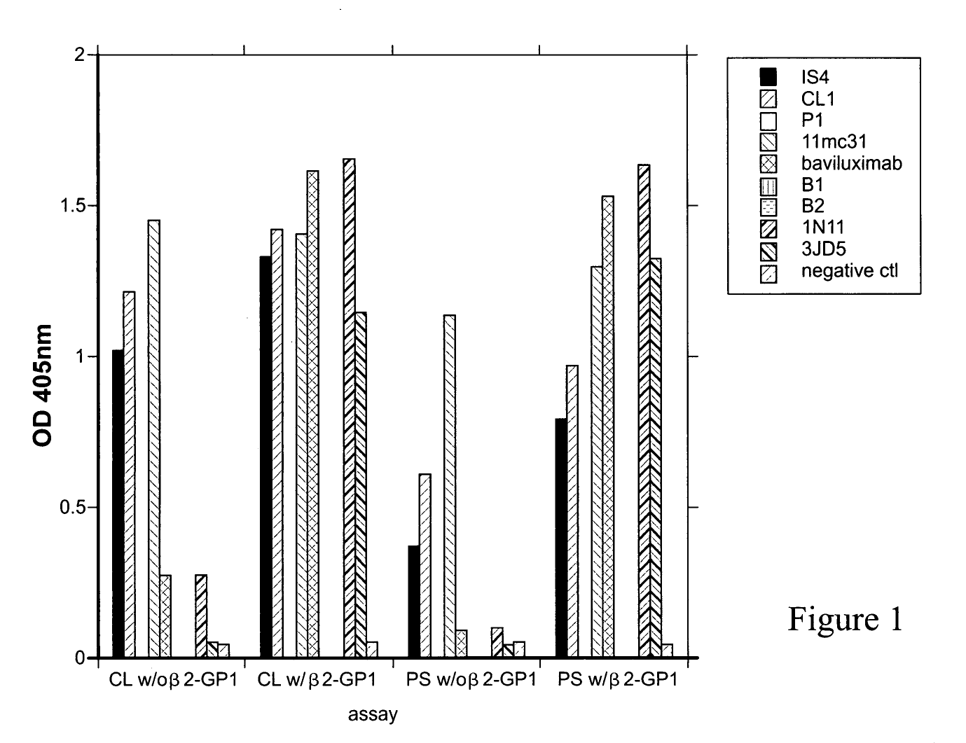

The most distinctive feature of the mAbs that inhibited HIV-1 infectivity versus those that did not was the absence of dependence of the inhibitory mAbs on β2-glycoprotein I (β2-GP 1) for binding to lipids. The four mAbs that inhibited HIV-1 did not require β2-GP1 for binding to CL or PS (P1, 11.31 (PGN 632), CL1, IS4) while the five mAbs that did not inhibit HIV-1 required β2-GPI for lipid binding (Table 3) (FIG. 1).

Anti-lipid mAb breadth of virus infectivity inhibition. The breadth of neutralization of 11.31 (PGN 632), P1, IS4 and CL1 mAbs was next determined. Of seven R5 viruses tested, the infectivity of all seven was inhibited by each of the four mAbs (Table 5). X4 viruses was tested, however, none of 4×4 viruses were inhibited by the 4 lipid antibodies (Table 4 and not shown). Similarly when the mAbs were tested against the R5SHIV SF 162P3, the infectivity of this SHIV was potently inhibited by all 4 mAbs, with the greatest inhibition seen with 11.31 (PGN 632) at 0.06 μg/ml IC80. However, the dualtropic R5/X4 SHIV 89.6P was not neutralized by any anti-lipid antibody.

TABLE 5
Breadth of HIV-1 infectivity inhibition
of four anti-lipid monoclonal antibodies.

| | Mabs | | | | | | |
|---|---|---|---|---|---|---|---|
| | | | | | anti- | Tri- | |
| HIV-1 | IS4 | CL1 | P1 | 11.31 | RSV | Mab | co- |
| isolates | (IC80 values in μg/mL) | | | | | | receptor |
| B.TORNO | 0.6 | 0.7 | 17 | 0.09 | >50 | 0.03 | CCR5 |
| B.PVO | 0.4 | 0.2 | 4.5 | 0.03 | >50 | 0.64 | CCR5 |
| B.6535 | 0.07 | 0.4 | 30 | 0.14 | >50 | >25 | CCR5 |
| C.DU123 | 0.06 | 0.2 | 1.7 | <0.02 | >50 | >25 | CCR5 |
| C.DU156 | 2.8 | 2.6 | 16 | 0.06 | >50 | >25 | CCR5 |
| C.DU151 | 1.8 | 4.1 | 0.1 | <0.02 | >50 | >25 | CCR5 |
| C.DU172 | 1.1 | 0.6 | 0.55 | <0.02 | >50 | >25 | CCR5 |
| SHIV 162P3 | 5.2 | 1.2 | 1.6 | 0.06 | >50 | 1.5 | CCR5 |
| SHIV 89.6P | >50 | >50 | >50 | >50 | >50 | >25 | CXCR4/CCR5 |
| SIV mac239 | >50 | >50 | >50 | >50 | >50 | >25 | |

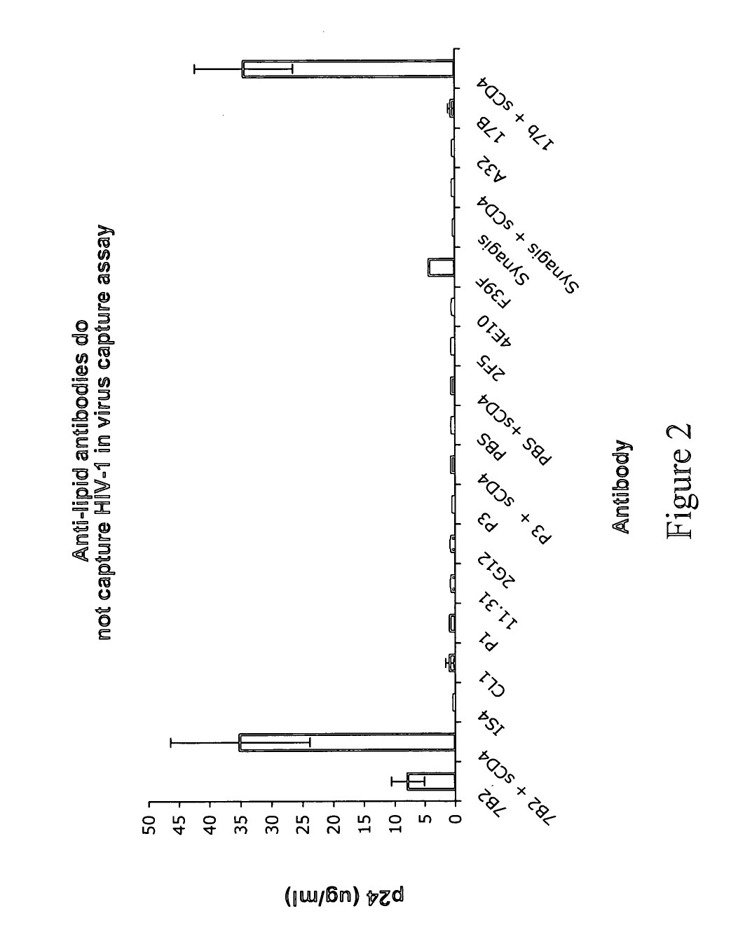

Lack of anti-lipid antibodies to capture HIV-1 virions. Anti-lipid, anti-HIV-1, and control mAbs were coated on microtiter plate wells and then incubated with primary isolate virions produced in PBMC in the presence or absence of soluble CD4. As expected, the anti-HIV-1 gp41 immunodominant region mAb 7B2 and the anti-gp120 V3 loop mAb F39F were able to capture HIV-1 virions. In addition, the anti-gp120 CCR5-binding site mAb 17b was able to capture virions in the presence but not in the absence of triggering by soluble CD4. In contrast, none of the anti-lipid mAbs were able to capture virions (FIG. 2).

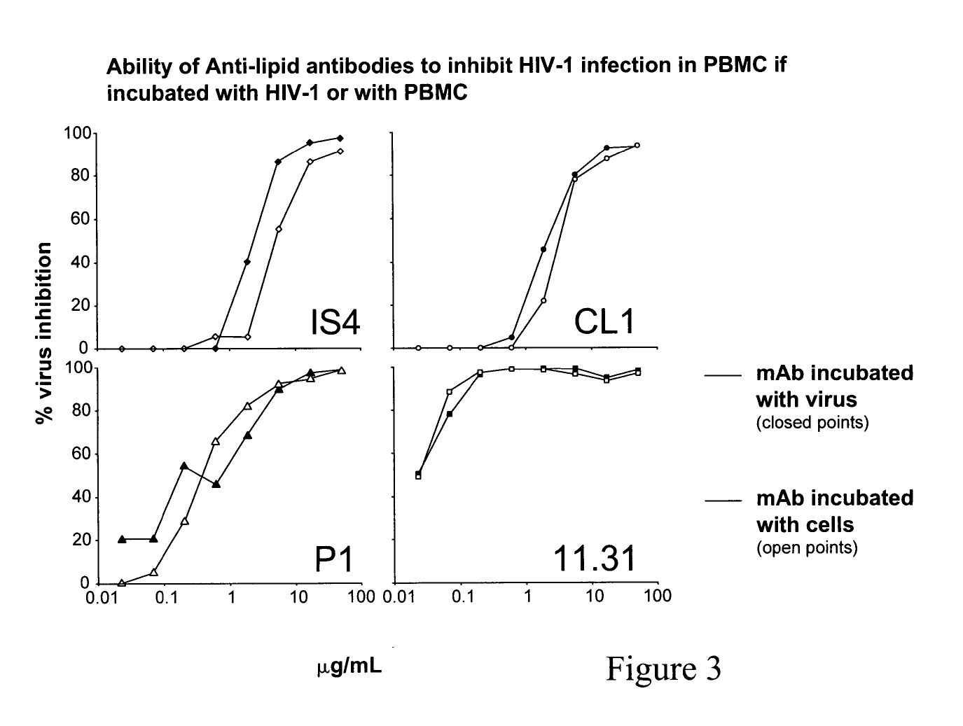

Site of inhibition effect of anti-lipid antibodies. Two assay protocols were studied to determine where in the PBMC cultures the mAbs were acting to inhibit HIV-1 infectivity. First, the mAbs were preincubated with virus for 60 min. prior to addition of virus-antibody mixture to phytohemagglutinin (PHA) activated PBMC. Second, anti-lipid mAbs were added first to PHA-activated PBMC X1 hour, then the PBMC washed and virus added to PBMC. In both circumstances, it was found the potency of mAb neutralization was found to be equal (FIG. 3), indicating that the anti-lipid antibodies inhibit HIV-1 infectivity by binding to the surface of target PBMC.

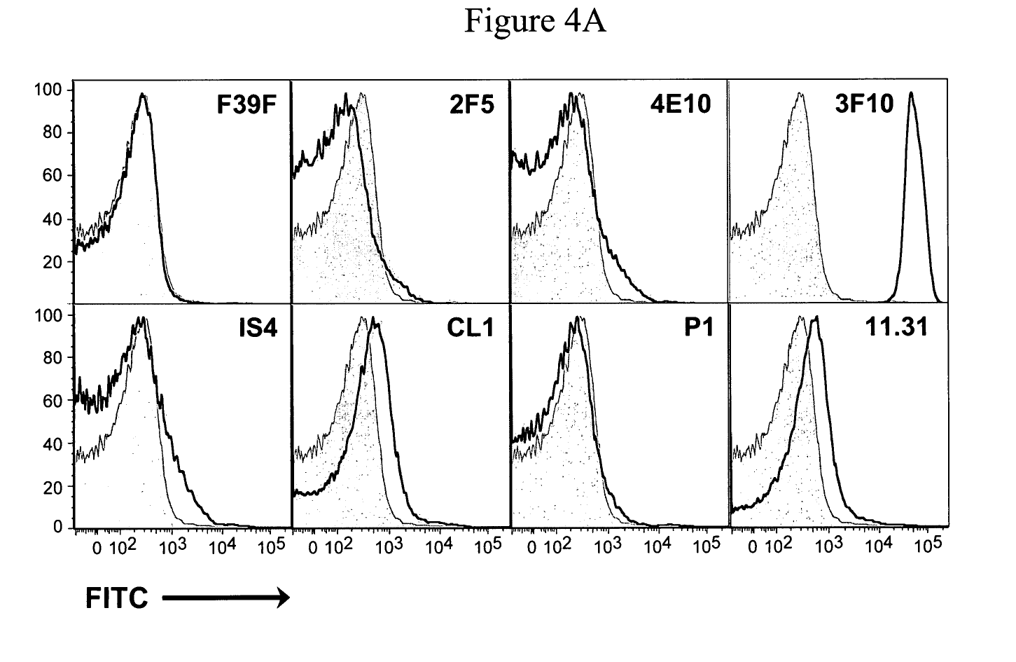

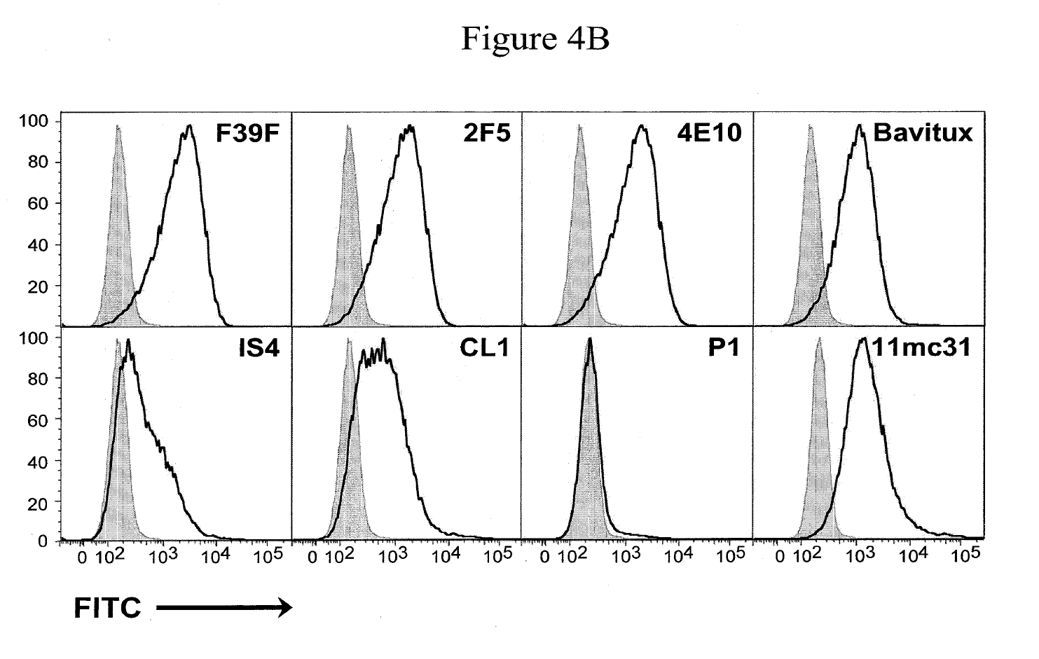

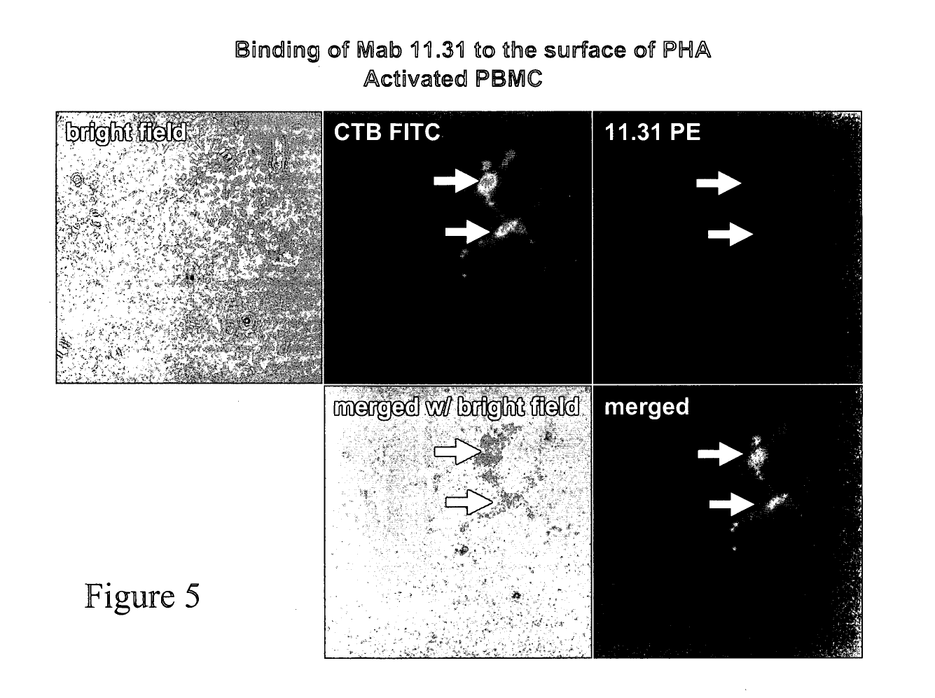

It was then asked if mAbs 11.31 (PGN 632), P1, IS4 and CL1 could bind to the surface of PHA activated PBMC. Analysis of the ability of anti-lipid mAbs to bind to PHA-activated PBMC (FIG. 4A) and HIVMN infected human H9 T cells (FIG. 4B) by flow cytometry showed that indeed a subset of PBMC and HIV-infected H9 T cells bound the anti-lipid antibodies. HIV-1MN infection of H9 T cells upregulated lipid mAb binding to viable infected cells to varying degrees depending on the mAb, with 11.31 (PGN 632)>CL1>IS4>P1 (FIG. 4B) and this correlated with the potency of the anti-lipid antibodies to inhibit HIV-1 infectivity. Moreover, 11.31 (PGN 632) mAb was found to bind to the surface of PHA activated PBMC in indirect immunofluoresence assays (FIG. 5).

To rule out that the anti-lipid antibodies were not reacting with HIV-1 Env, surface plasmon resonance analysis of anti-lipid antibody reactivity with a series of recombinant Env oligomers was performed. Whereas 2F5 and 4E10 bound well to JRFL and CON—S gp140 oligomers, none of the anti-lipid antibodies bound to HIV-1 Env (not shown). Moreover, as mentioned, the lipid antibodies did not capture HIV-1 virions (FIG. 2).

To determine the stage of HIV-1 infection that the mAbs inhibited, a timing study was performed of addition of the mAbs at the time of addition of the virus, and at 24, 48, and 72 hours after adding virus to PBMC. It was found that, for each of the antibodies, neutralization was observed at the later time points (Table 6). For all antibodies, the neutralization was attenuated at the later time points and correlated with the initial potency of the antibody. Significantly, both CL1 and 11.31 (PGN 632) were able to neutralize when added 48 hours after the start of the infection with IC80s of 0.22 and 0.07 μg/mL, respectively.

TABLE 6
Effect of time of introduction on the inhibitory effect of
anti-lipid antibodies against B.6535 in the PBMC assay.

| | Mabs | | | | |
|---|---|---|---|---|---|
| time after | IS4 | CL1 | P1 | 11mc31 | Synagis |
| infection | (IC80 values in μg/mL) | | | | |
| 0 h | 0.91 | 0.19 | 2.4 | <0.02 | >50 |
| 24 h | >50 | 0.60 | >50 | 0.18 | >50 |
| 48 h | >50 | 0.22 | >50 | 0.07 | >50 |
| 72 h | >50 | >50 | >50 | >50 | >50 |

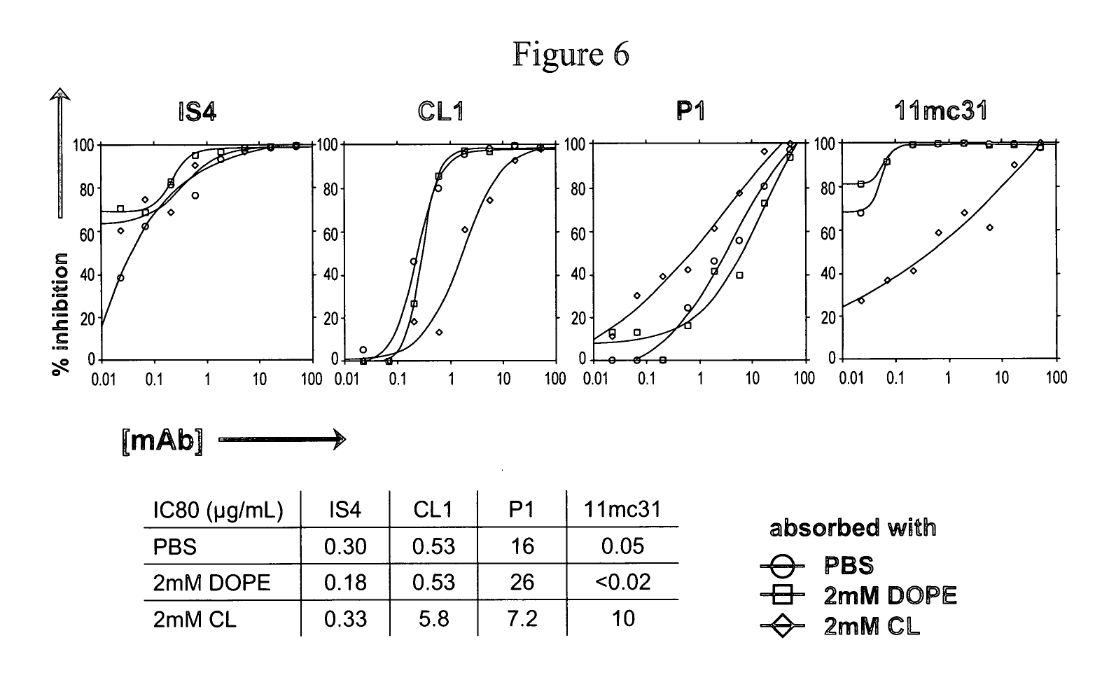

Neutralization activity of anti-lipid antibodies is altered by preincubation with lipids. To investigate the specificity of these antibodies, neutralization assays were performed with mAbs preincubated with PBS, 2 mM cardiolipin (CL) or 2 mM dioleoylphosphatidylethanolamine (DOPE) (FIG. 6). When tested against B.6535, two of the antibodies, CL1 and 11.31 (PGN 632), showed no change in potency when incubated with DOPE but did show a loss of potency following incubation with CL, with a reduction in IC80 by 11-fold and 200-fold, respectively. IS4 showed little change upon incubation with either lipid while P1 showed a 1.6-fold decrease in potency with preincubation with DOPE. These data are consistent with the target of these antibodies being lipids or molecules associated with lipids on target PBMC. The change in inhibition by lipid preincubation also correlated with the potency of the unabsorbed antibodies.

Direct ligation of target cell PS results in virus inhibition. β2-GP-1-Fc dimer is a construct of two full length (domains I through V) molecules of β2-GP-1 joined by an IgG1 Fc. β2-GP-1 binds to PS and is the target for many pathogenic antibodies in patients with primary or secondary forms of APAS (DeGroot et al, J. Thromb. Haemost. 3:1854-1860 (2005)). Thus, if a dimer of β2-Gp-1 could inhibit HIV-1 infectivity, it would provide direct evidence of the requirement for binding PS in HIV-1 infectivity inhibition in PBMCs. Indeed, while not as potent as the anti-lipid antibodies, β2-GP-1 inhibited B.6535, C.DU123, and SHIV SF162P3 at IC80s of 12, 1.4, and 29 μg/mL, respectively.

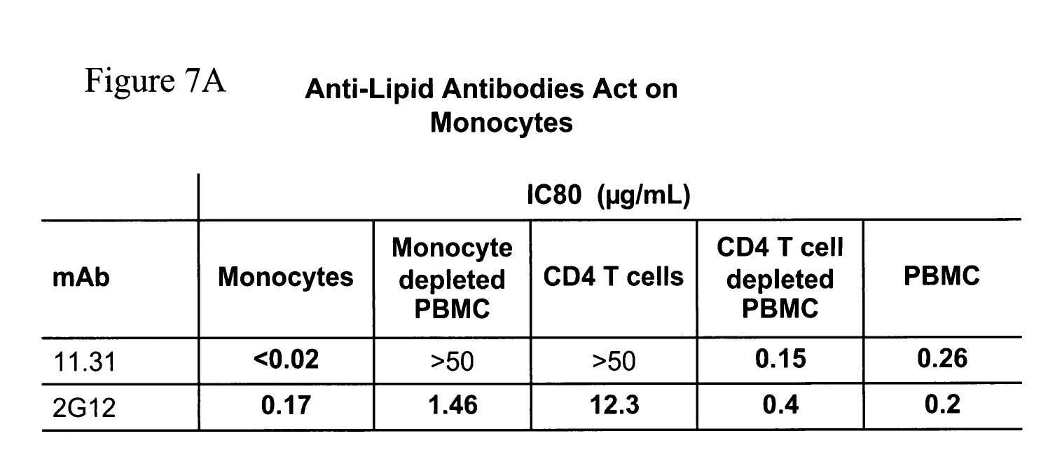

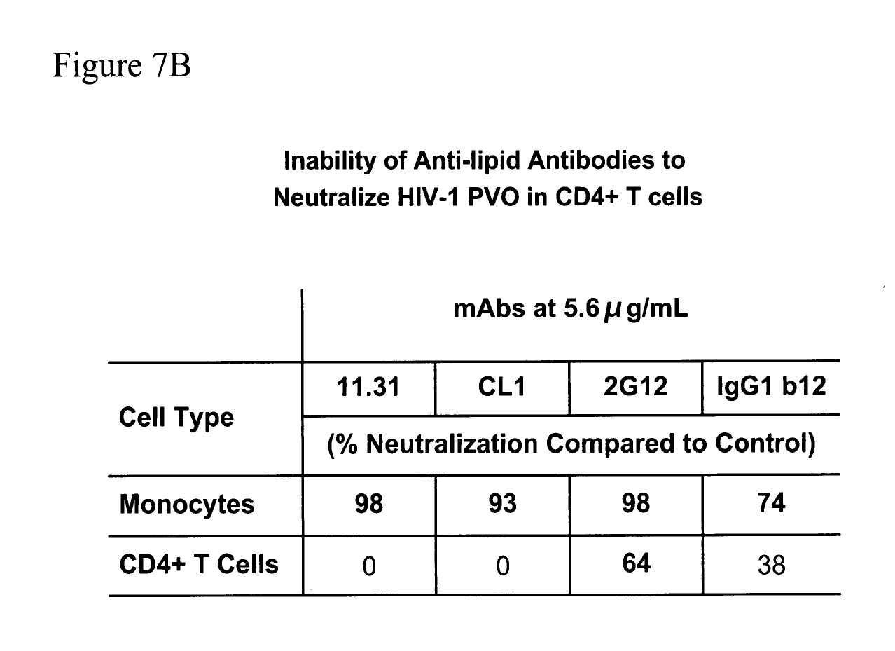

Incubation of mAb 11.31 (PGN 632) with monocytes but not CD4+ PMBC T Cells prevents HIV-1 infection. Anti-lipid antibodies do not inhibit the HIV-1 infectivity of PB CD4+ T cells alone; rather anti-lipid antibodies only inhibit HIV-1 infectivity of PBMC cultures when monocytes are present. In contrast, anti-HIV-1 carbohydrate mAb 2G12 inhibits infectivity in purified CD4+ T cells regardless of whether monocytes are present or not. (See FIG. 7.)

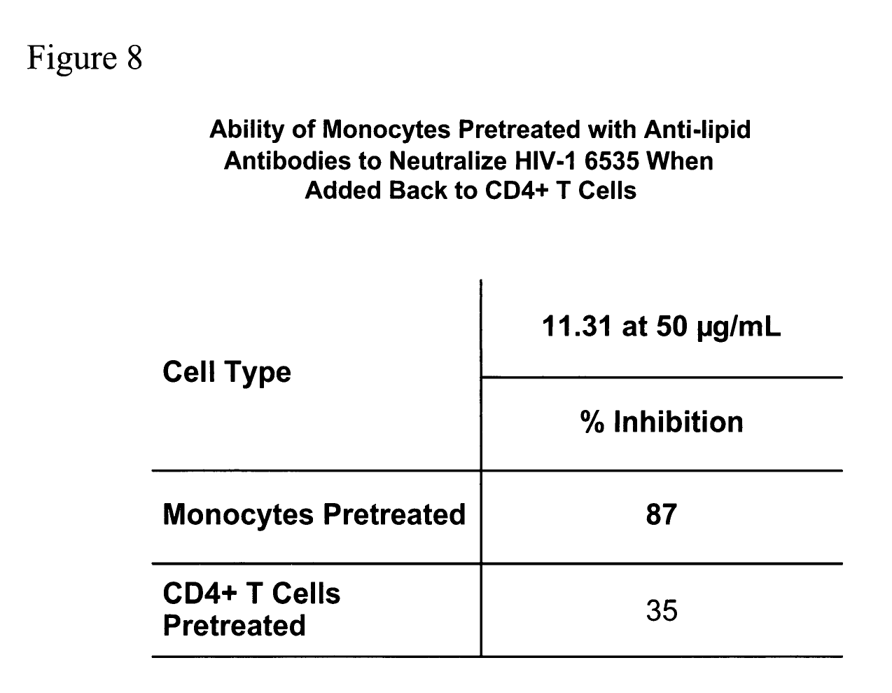

Anti-lipid antibodies, when coated on PB monocytes, and the antibody-coated PB monocytes are added back to CD4+ T cells, now inhibit the infectivity of purified PB CD4+ T cells. In contrast, when purified PB CD4+ T cells are pretreated with anti-lipid antibody and added back to CD4+ T cells, no ability of the antibody-treated PB CD4+ T cells to inhibit HIV-1 infectivity of CD4 T cells is seen. Thus, it was surmised that the lipid antibodies must be stimulating some activity from monocytes that had a specific inhibiting effect on HIV-1 infectivity. (See FIG. 8.)

Ability of anti-lipid antibodies to induce CCR5 (R5) but not CXCR4 (X4)—binding chemokines from monocytes.

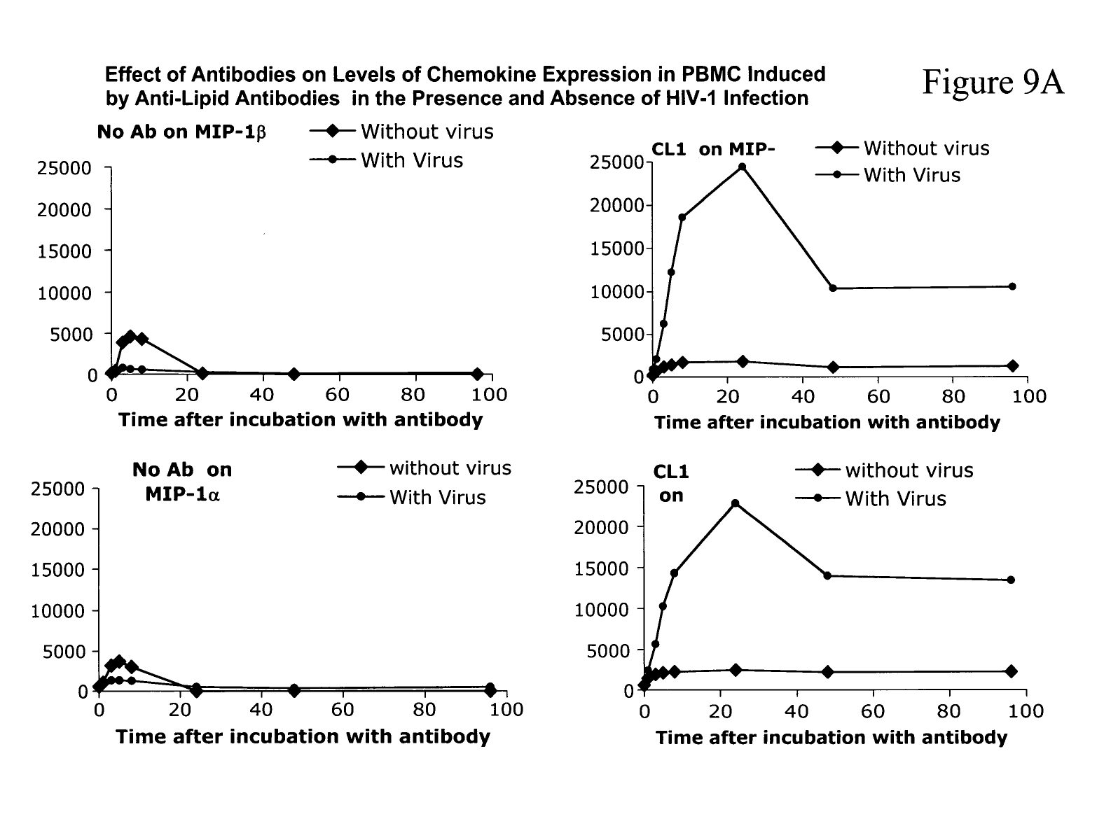

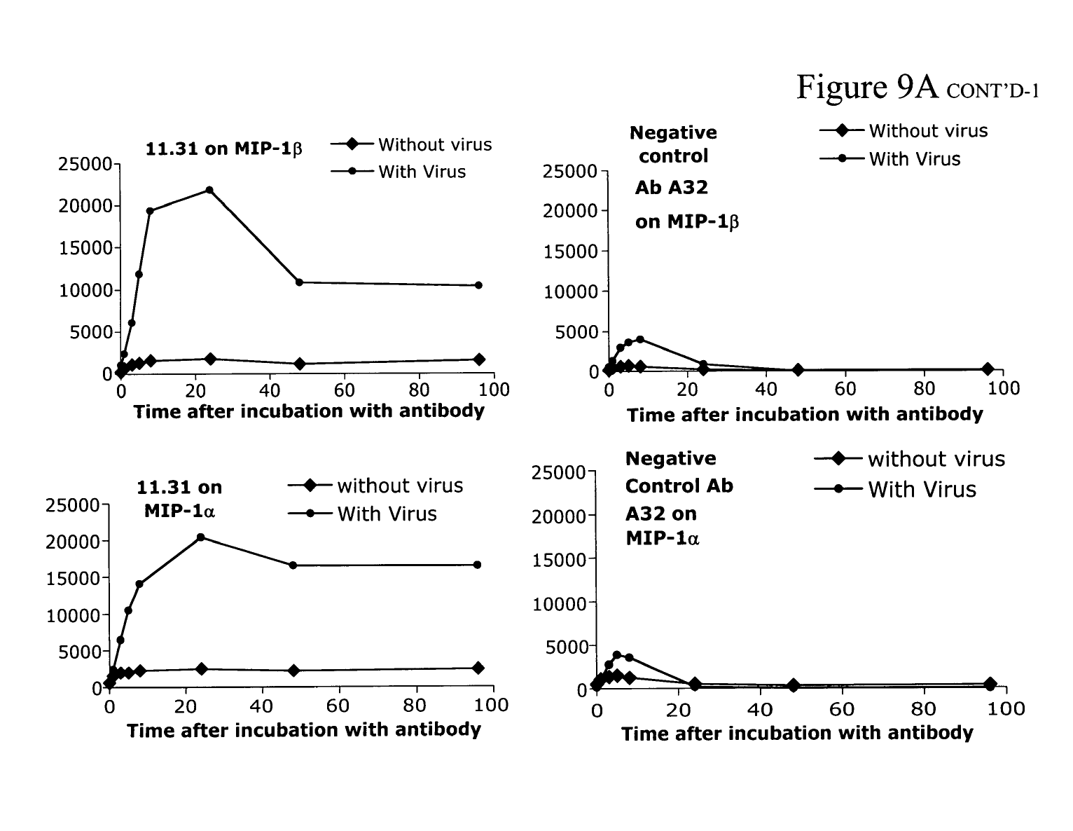

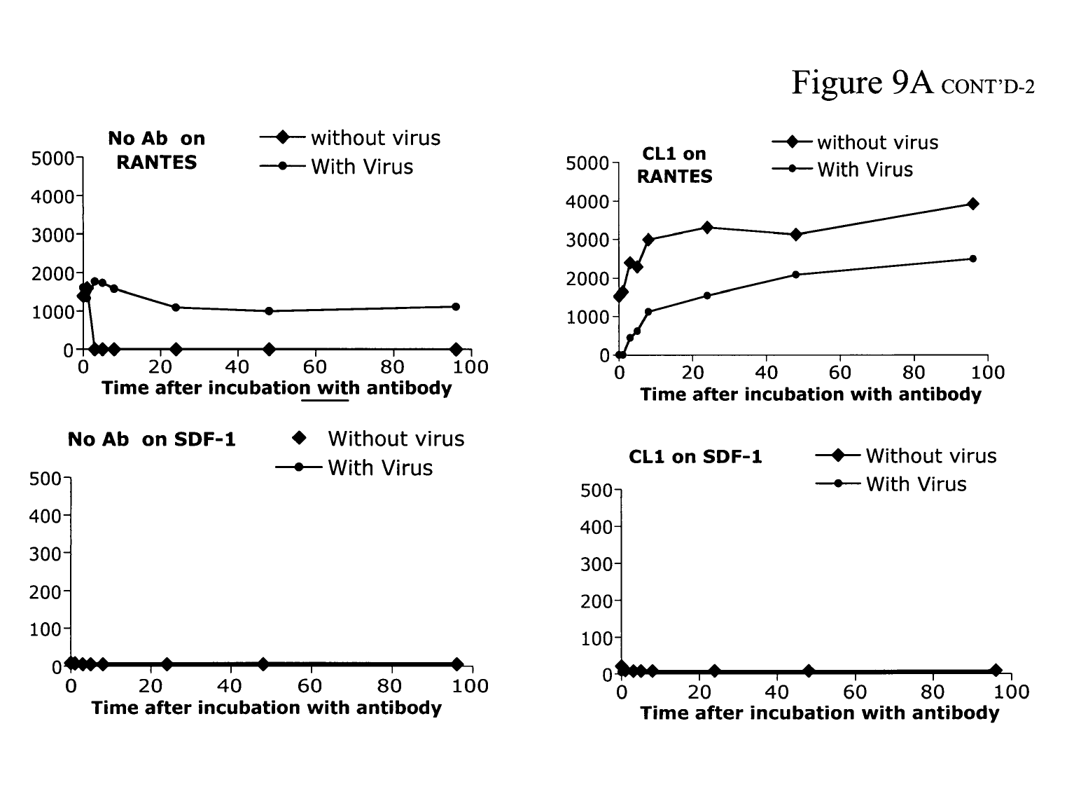

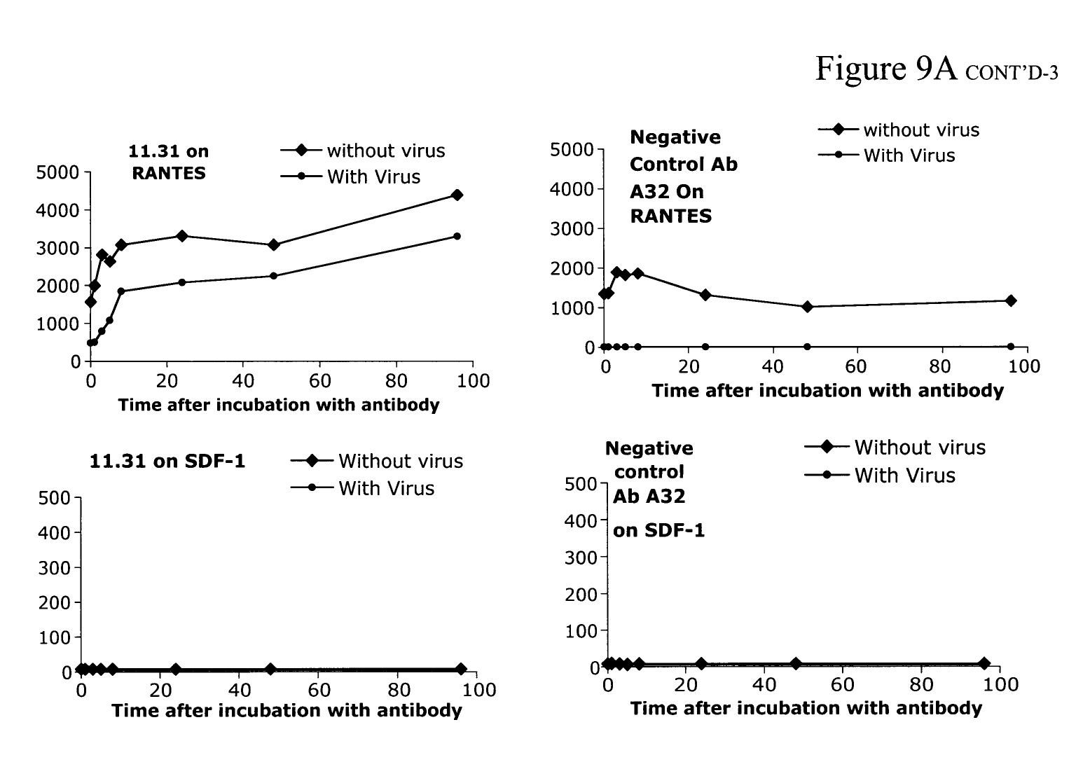

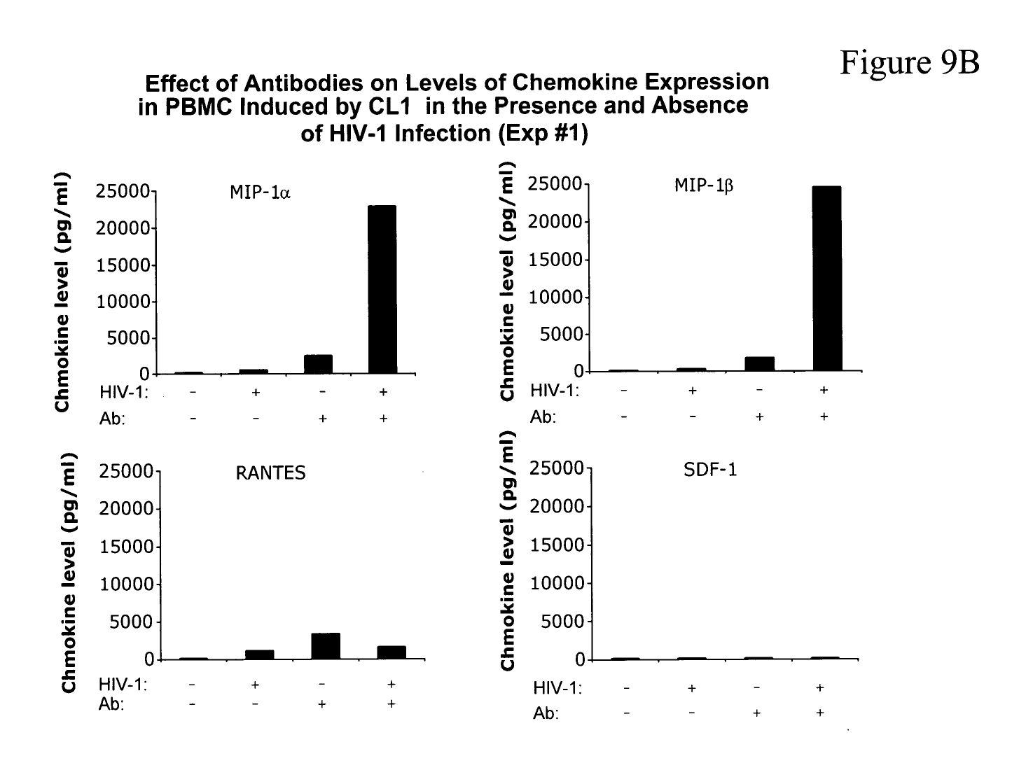

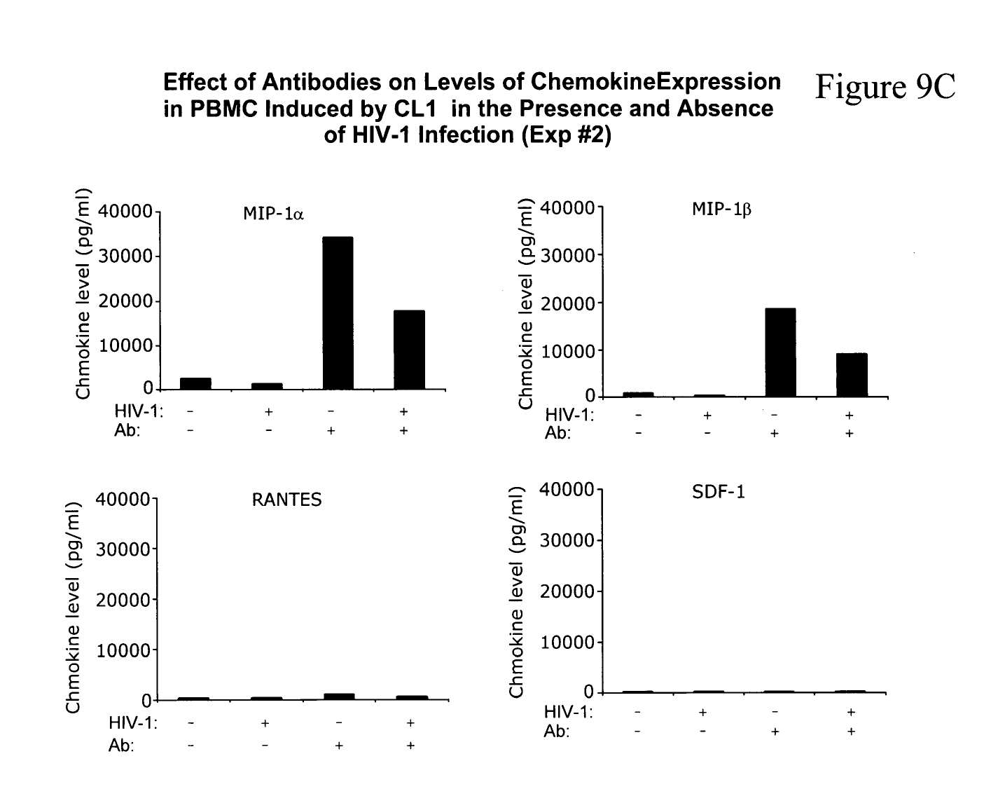

The next question asked was whether the anti-lipid antibodies could induce R5 but not X4 chemokines from monocytes. FIG. 9 shows that indeed this is the case. Monocytes in the presence of the anti-lipid antibodies and no virus induce the R5 chemokines MIP-1α, MIP-1β and RANTES but do not induce the X4 chemokine SDF-1. Moreover, HIV-1 virus added to monocytes has a small or minimal effect on R5 chemokine induction. However, in the presence of both antibodies and virus, the production of chemokines and, in particular, MIP-1α and MIP-1 β is enhanced. Thus, the mechanism of inhibition of the anti-lipid antibodies is via induction of R5 chemokines from monocytes (and likely other myeloid cells such as dendritic cells and tissue macrophages) by a combination of the anti-lipid antibodies and HIV-1. Moreover, this selective induction of R5 but not X4 chemokines by anti-lipid antibodies+HIV-1 is the explanation for why the anti-lipid antibodies inhibit the infectivity of only R5 chemokines but not X4 chemokines. Recently, Keele et al have demonstrated that HIV-1 transmitted viruses are virtually all R5 viruses (Keele, Brandon et al. Proc. Natl. Acad. Sci. 105:7552-7, Epub 2008 May 19 (2008)). Taken together, these data indicate that the anti-lipid antibodies such as 11.31 (PGN 632) and CL1 can be particularly effective as a treatment of early HIV-1 infection, and that they can be useful as a post-exposure prophylaxis of HIV-1 infection and able to protect against the R5 transmitted virus.

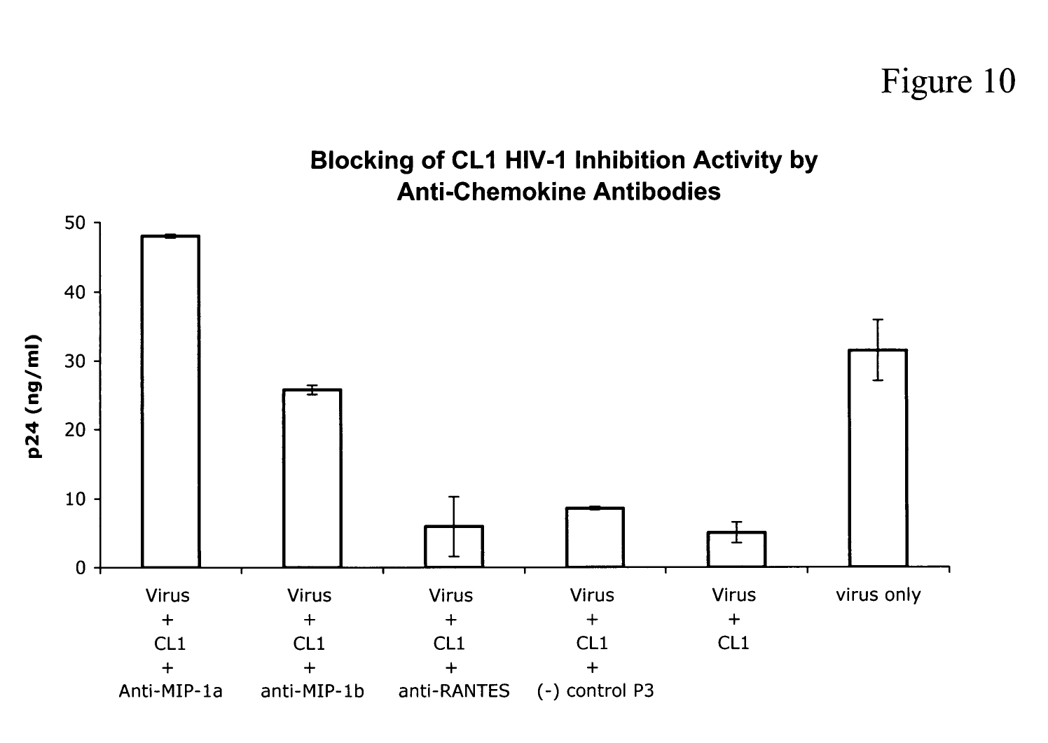

Ability of antibodies against R5 chemokines to inhibit the ability of anti-lipid antibodies to inhibit HIV-1 infection of PBMC. It was next asked if antibodies that neutralize the effects of R5 chemokines, when added to the PBMC HIV-1 infectivity assay, could inhibit the ability of mAbs 11.31 (PGN 632) and CL1 to inhibit PBMC infection by HIV-1 (FIG. 10). It was found that antibodies that neutralize the R5 chemokines MIP-1α and MIP-1β were the strongest inhibitor of the ability of the anti-lipid antibodies to inhibit HIV infectivity. Thus, indeed, the induction of R5 chemokines in the presence of HIV-1 by anti-lipid antibodies can inhibit HIV-1 infection of PBMC.

### Example 2

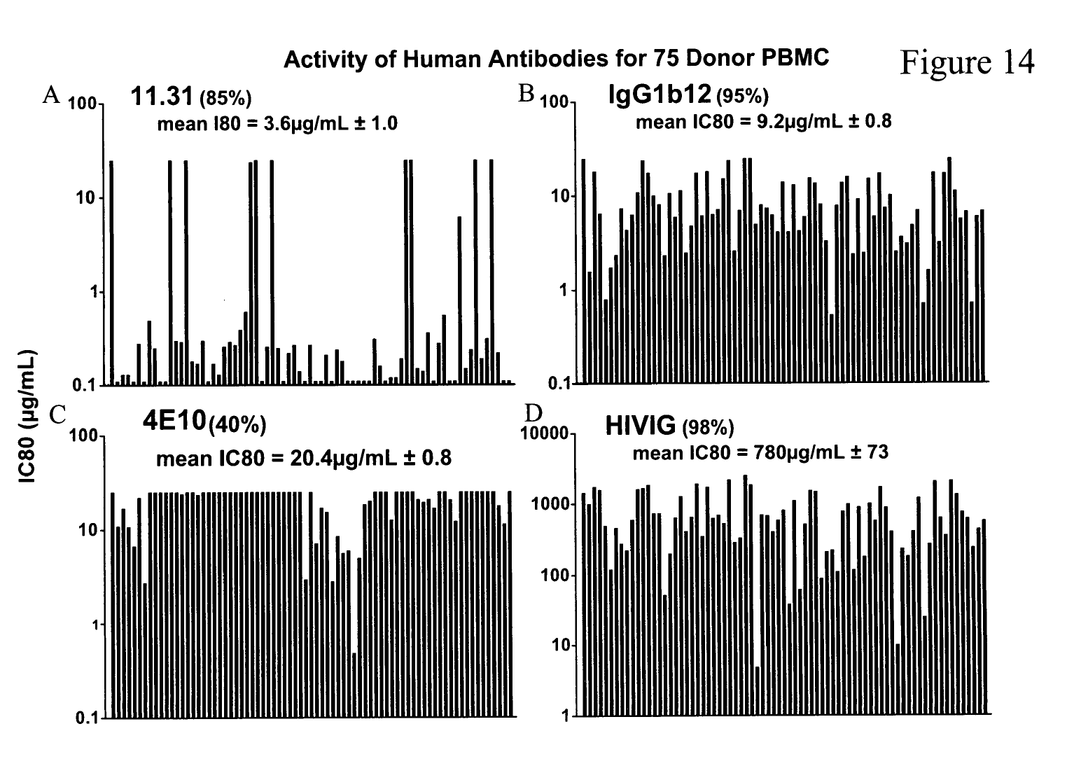

PBMC were obtained using standard methods from 75 healthy donors and used as targets in the PBMC assay with HIV-BaL.LucR.T2A.ecto/hPBMC as the infecting virus. Monoclonal antibodies 11.31 (FIG. 14A), IgG1b12 (FIG. 14B), and 4E10 (FIG. 14C), and the human polyclonal antibody preparation HIVIG (FIG. 14D), were tested for ability to inhibit infection. Data are plotted as concentration of antibody required for 80% inhibition of HIV-1 infection compared to a control infection without antibody. Each column represents data obtained from an individual donor PBMC preparation and correspond among the graphs. 11.31 demonstrated inhibition of infection in 85% of donor PBMC tested with a mean IC80 value of 3.6 μg/mL. IgG1b12 inhibited in 95% of PBMC tested with a mean IC80 of 9.2 μg/mL; 4E10 inhibited in 40% of PBMC tested with a mean IC80 of 20.4 μg/mL; HIVIG inhibited in 98% of tested PBMC with a mean IC80 of 780 μg/mL.

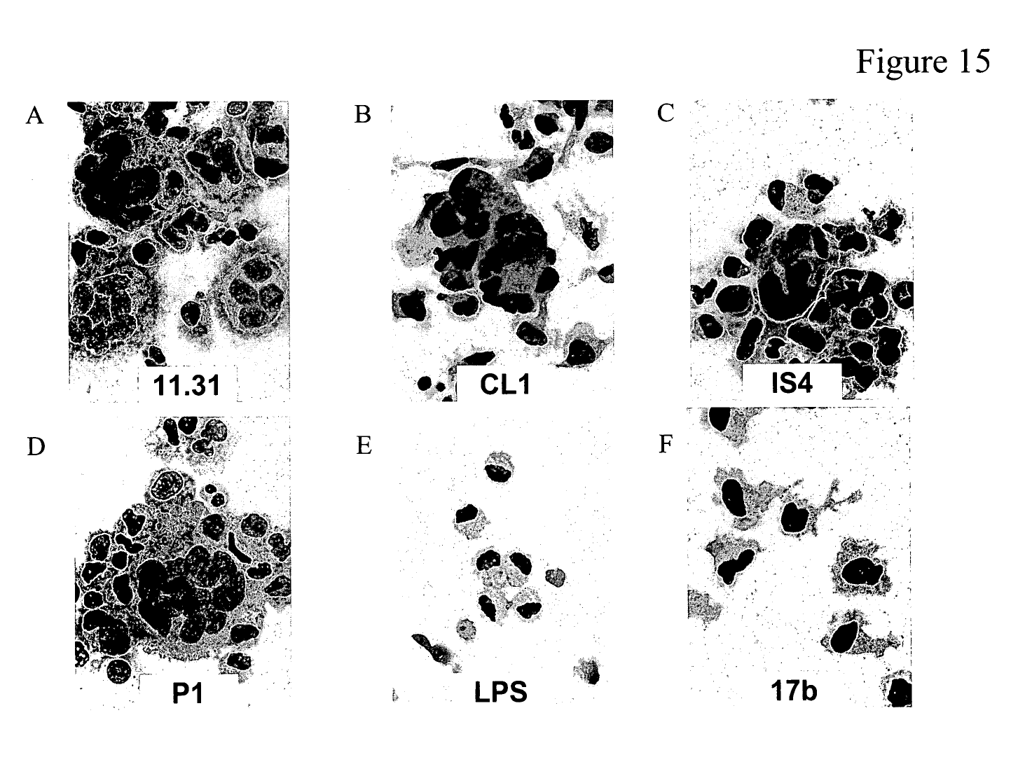

Monocytes obtained by elutriation from a healthy donor and at >94% purity were incubated in chamber slides or in 6-well plates in the presence of monoclonal antibodies (at 10 μg/mL final concentration), lipopolysaccharide (Sigma, final concentration 10 μg/mL), or no stimulus. After 96 hours of incubation the supernatants in the chamber slides were removed and the slides were Wright stained and then viewed under microscopy. After 7 days, cells in the 6-well plates were removed and spun onto cytoprep slides for staining. Incubation with monoclonal antibodies 11.31 (FIG. 15A), CL1 (FIG. 15B), and IS4 (FIG. 15C) after 96 hours induced the formation of multinucleated giant cells (polykaryons). Similarly, after 7 days, the antibody P1 (FIG. 15D) induced the formation of polykaryons. Stimulation with lipopolysaccharide (FIG. 15E), control antibodies 17b (FIG. 15F), or with palivizumab, A32, or F39F (not shown) did not induce polykaryon formation.

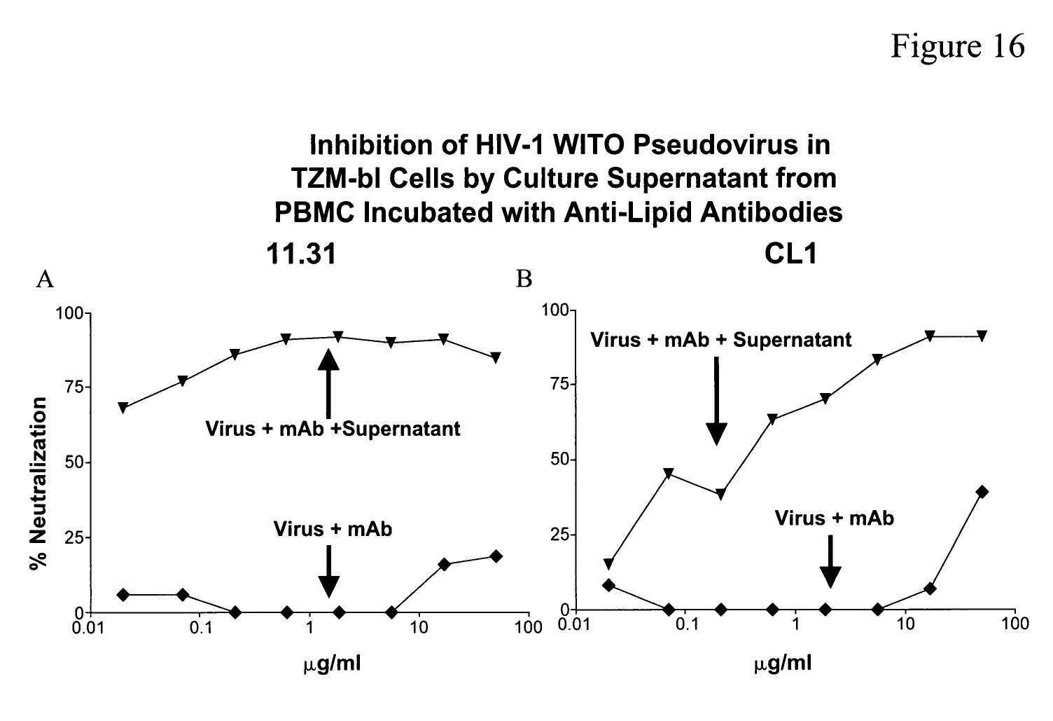

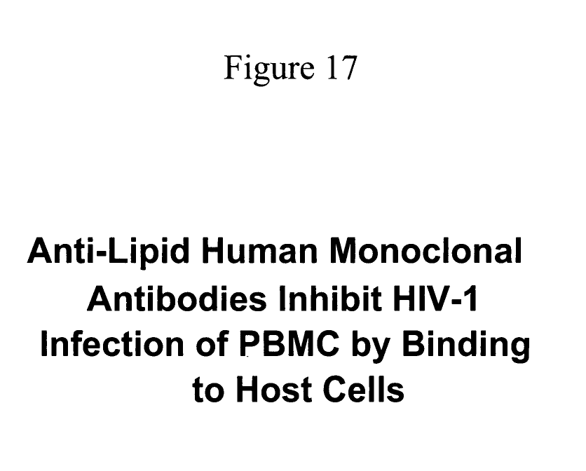

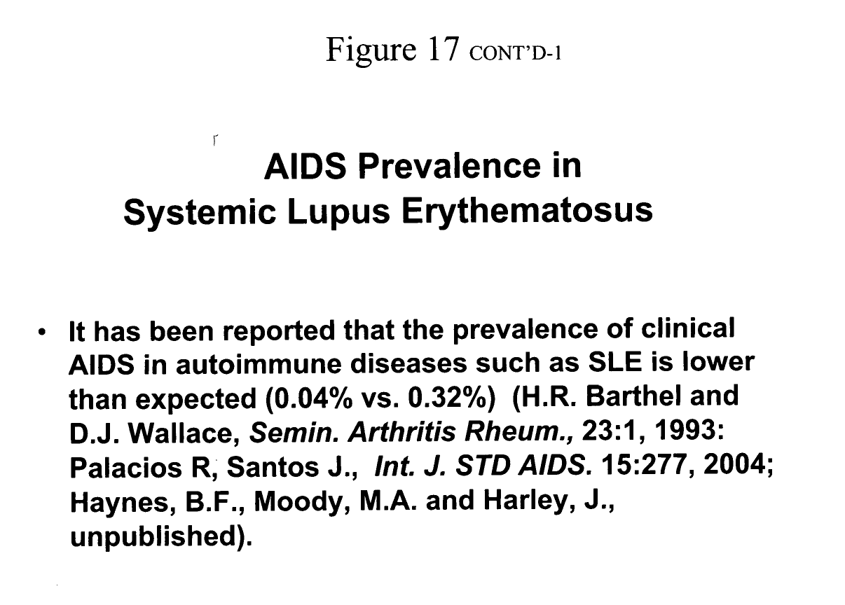

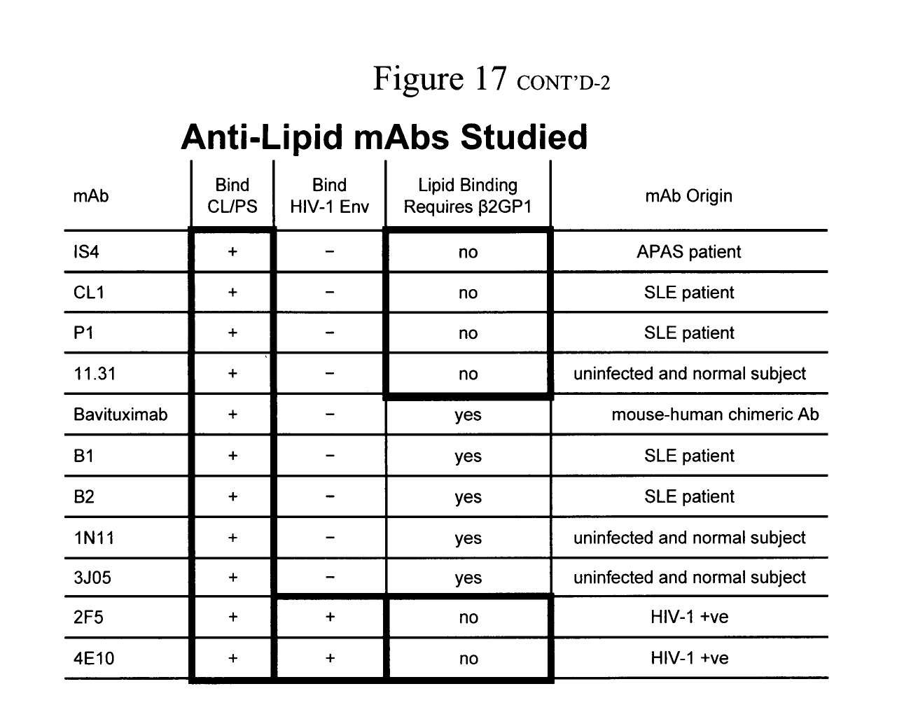

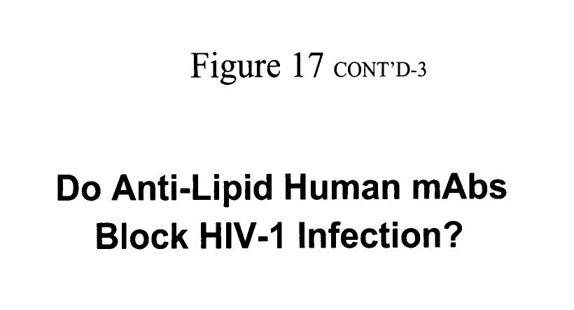

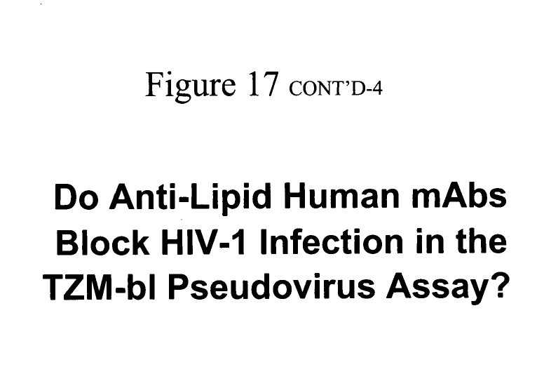

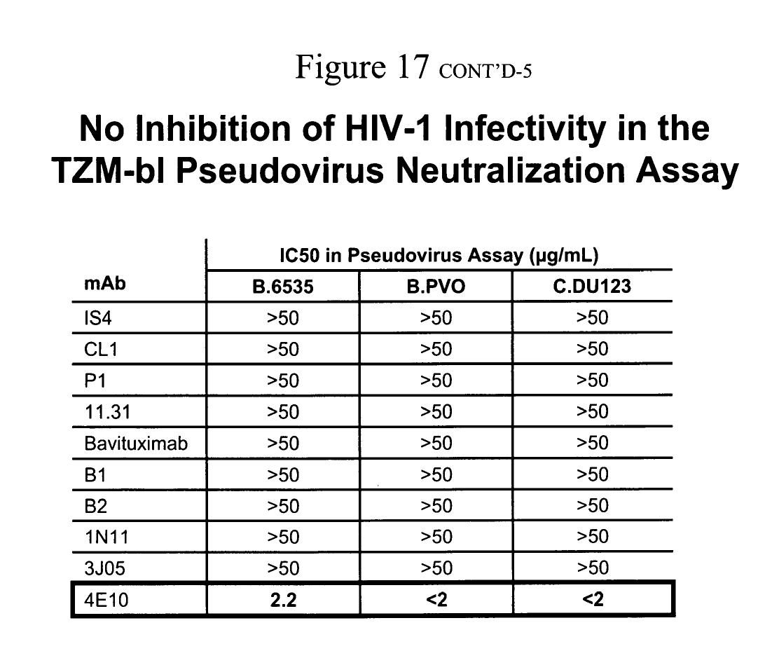

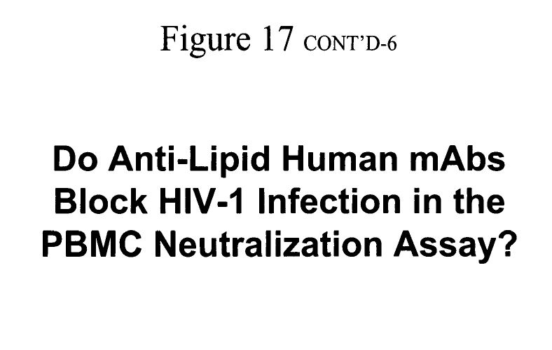

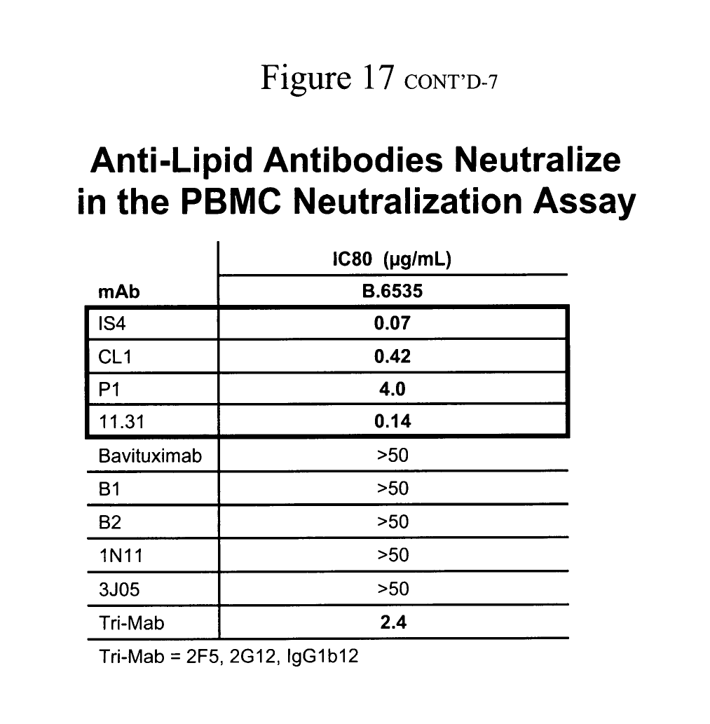

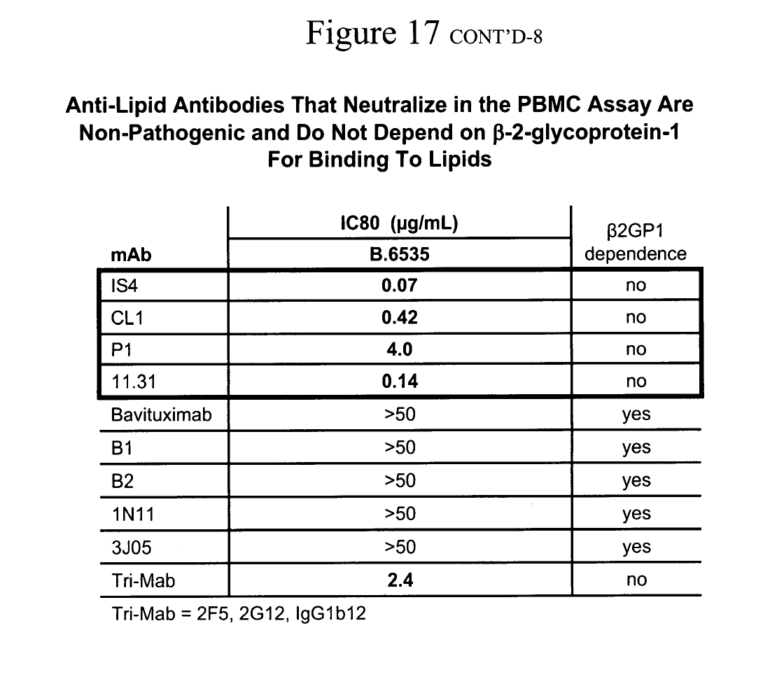

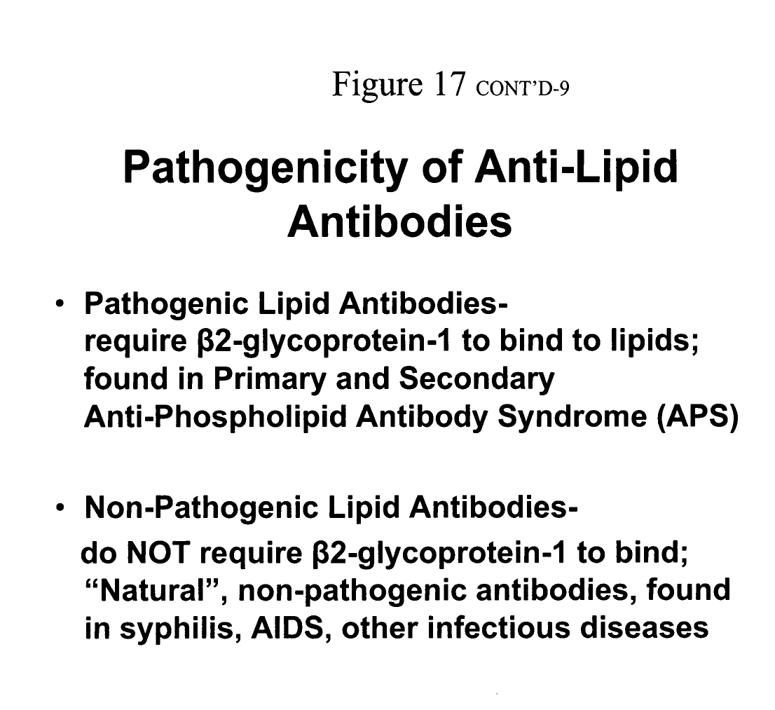

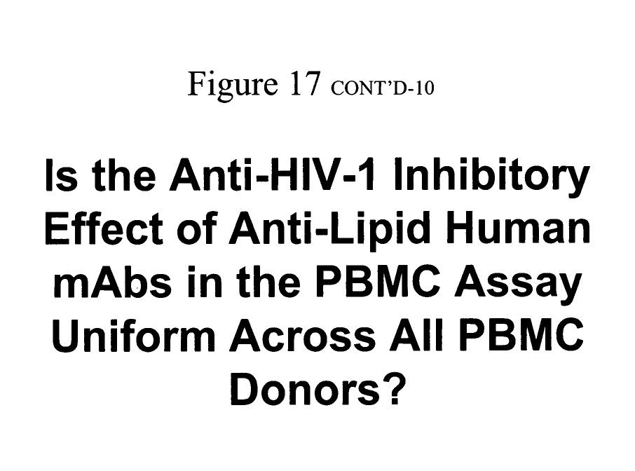

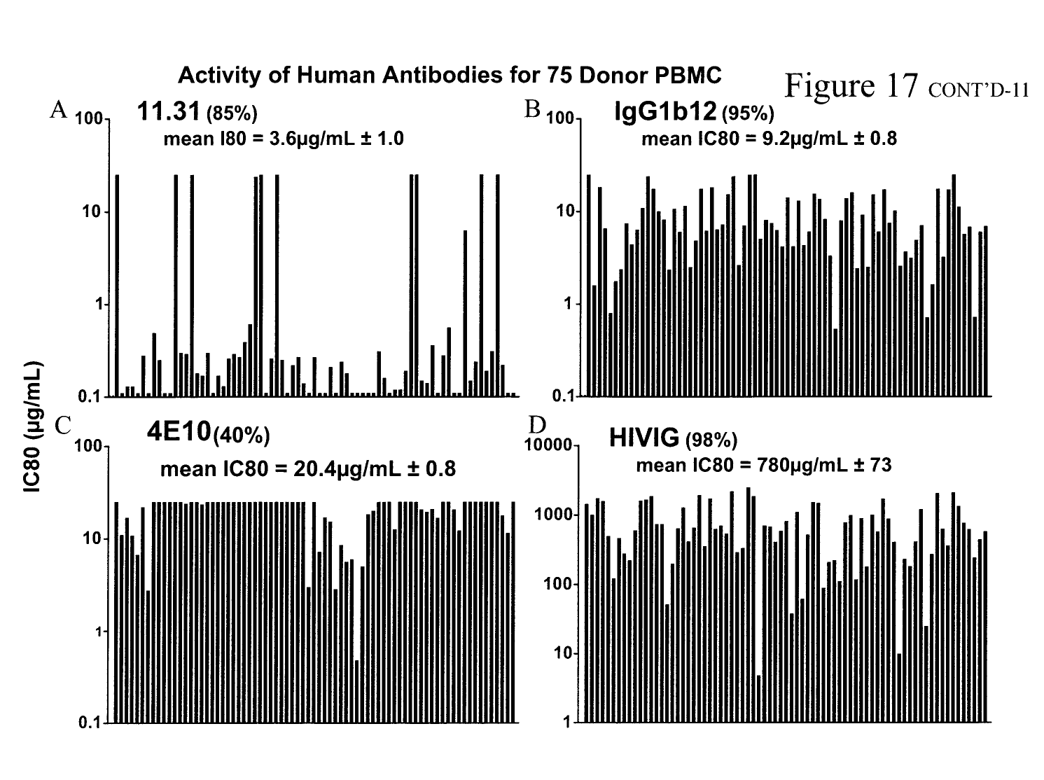

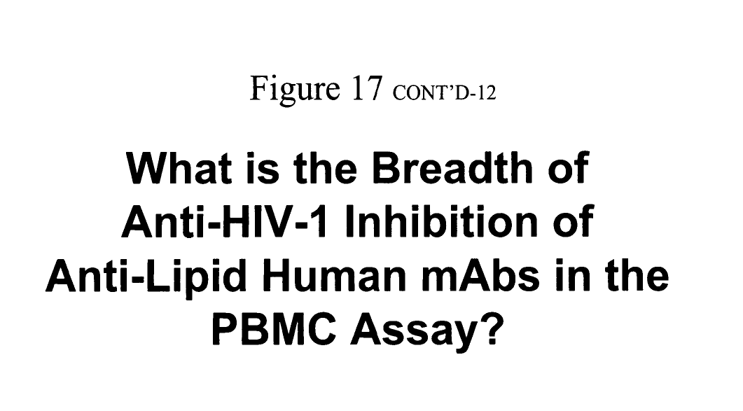

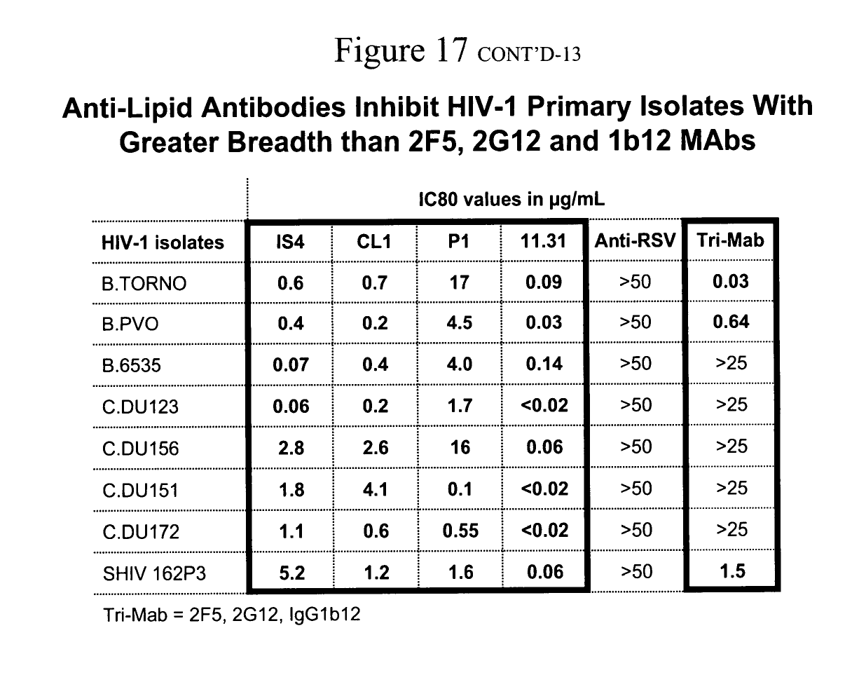

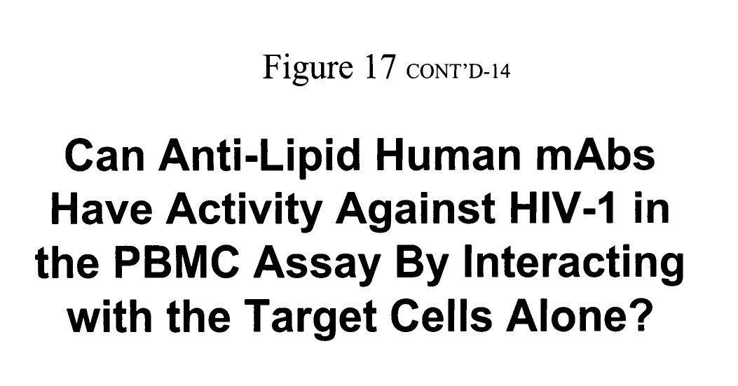

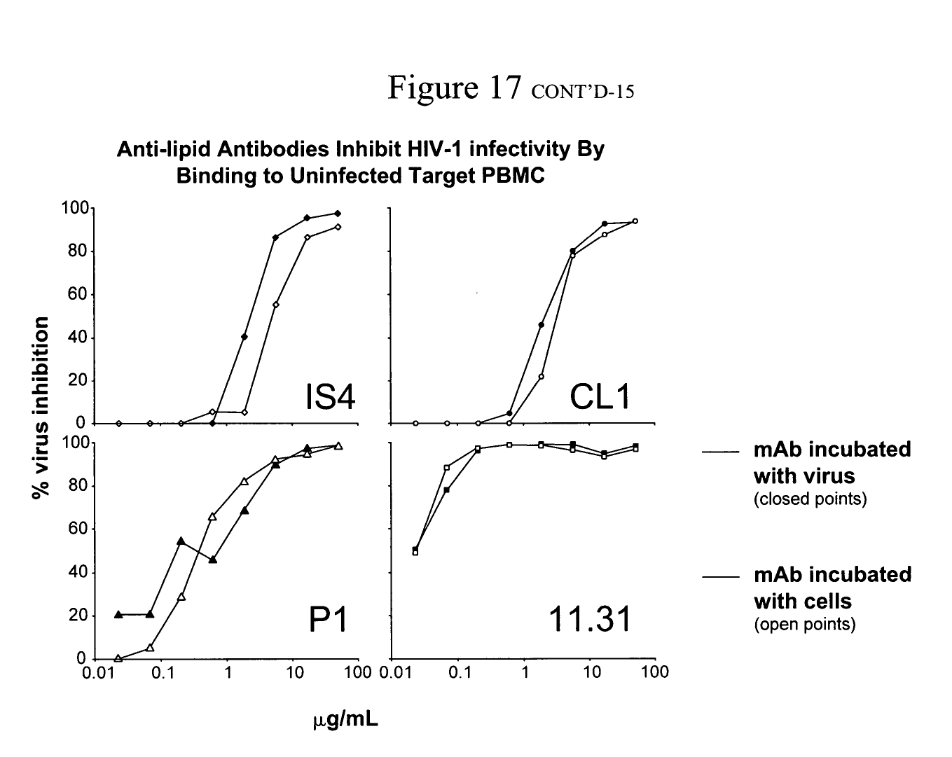

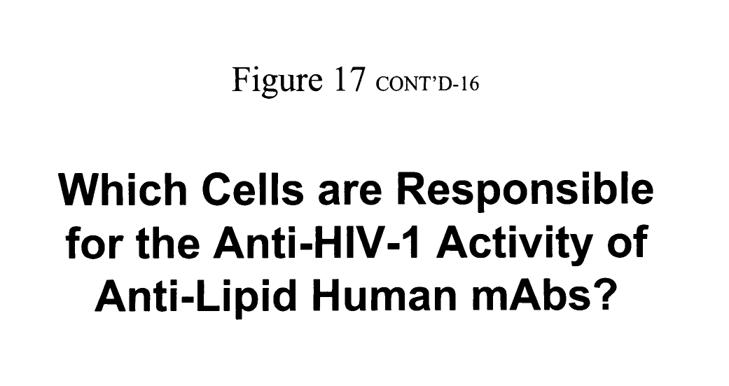

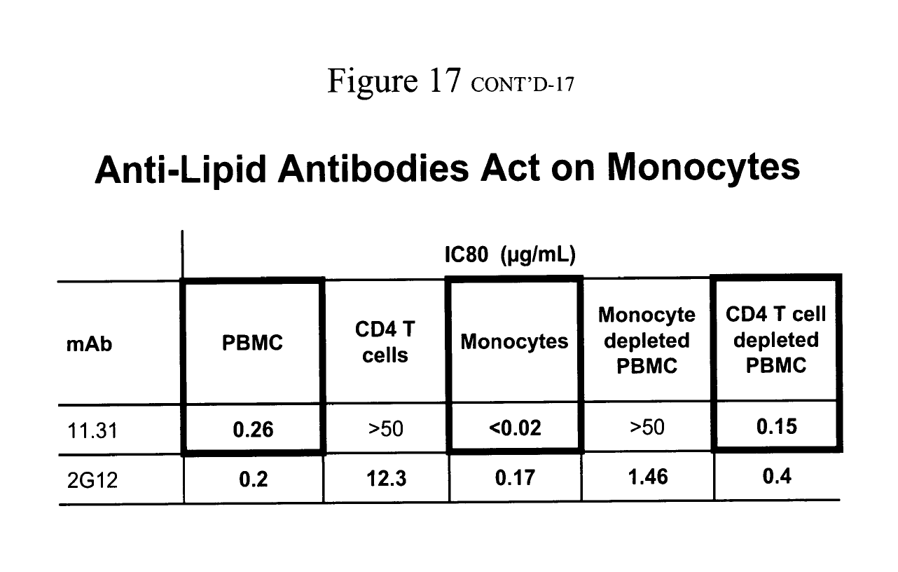

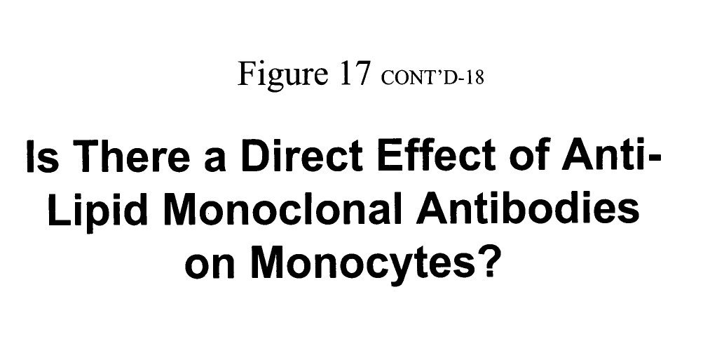

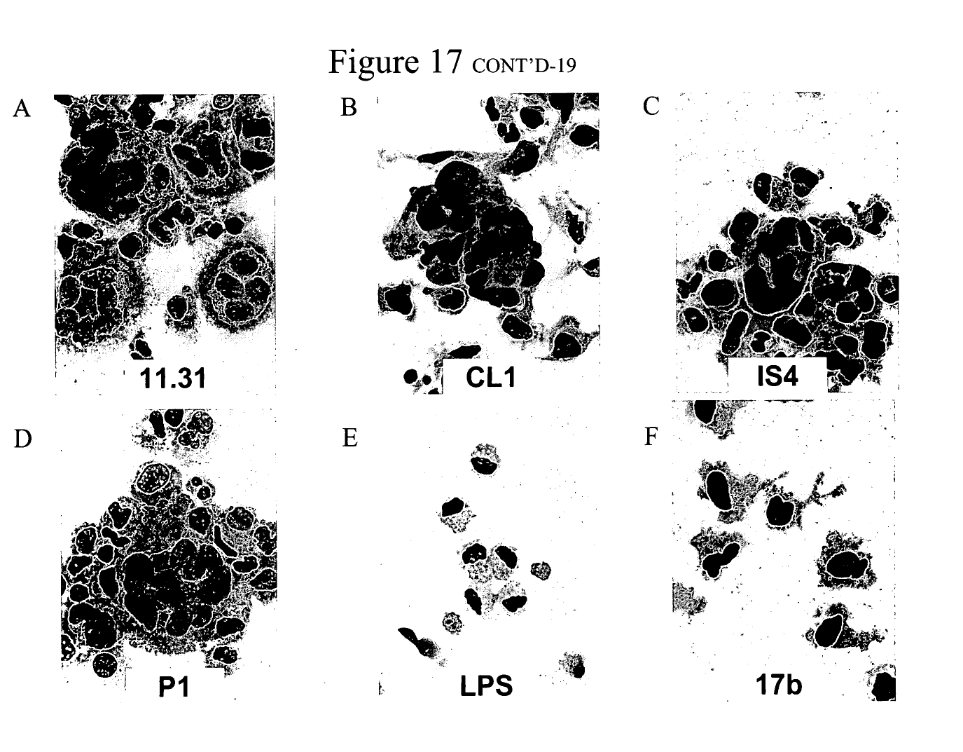

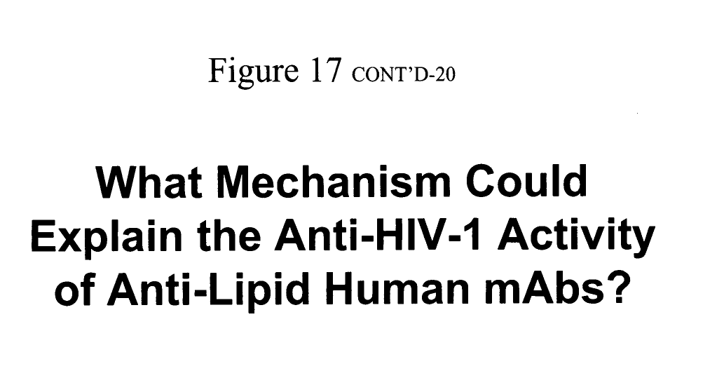

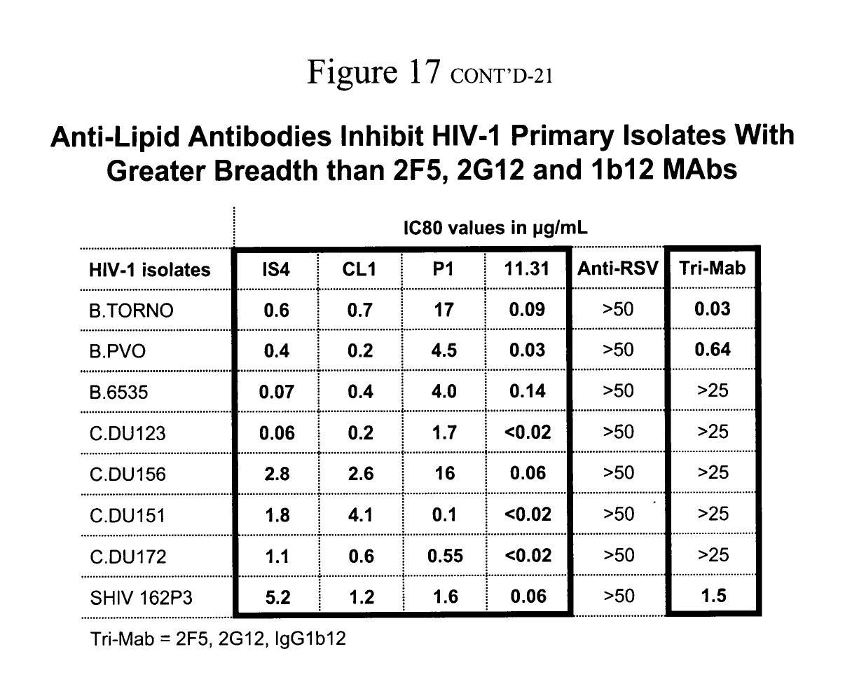

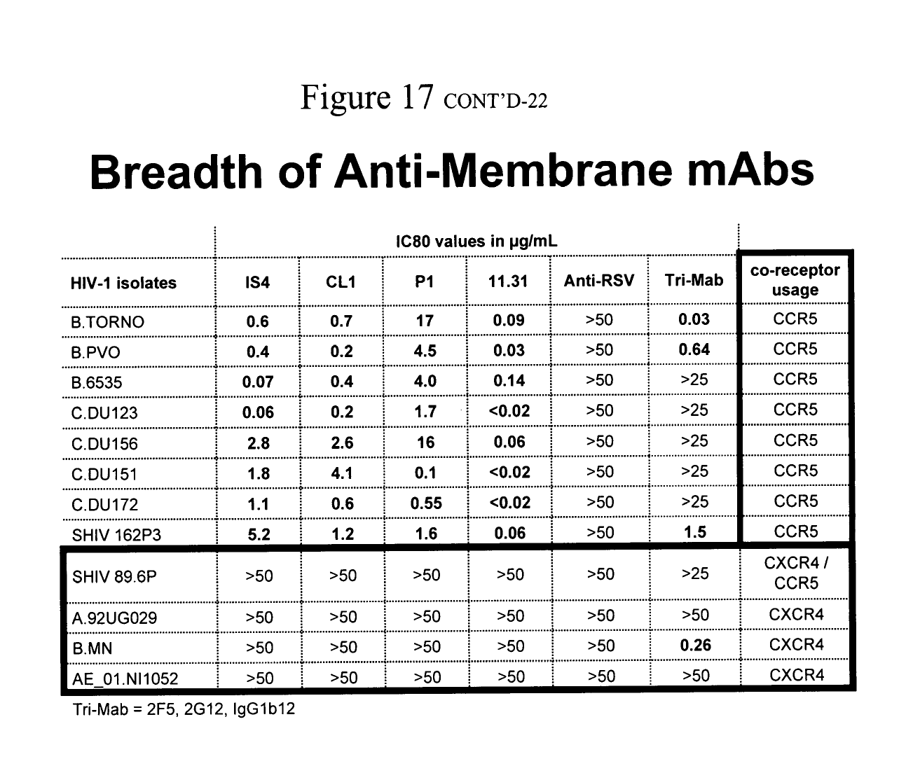

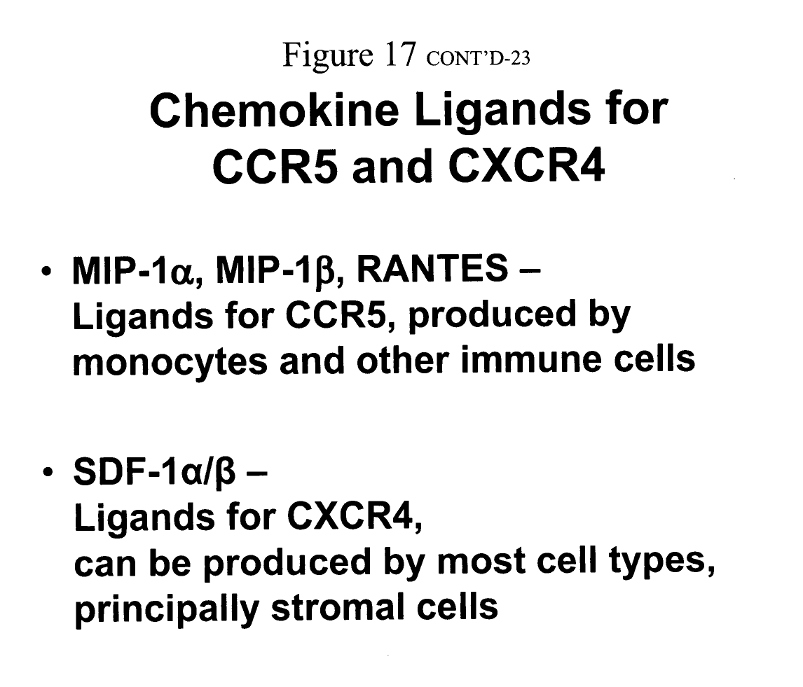

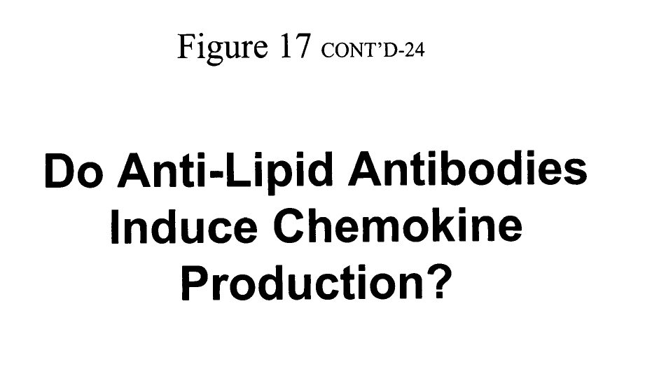

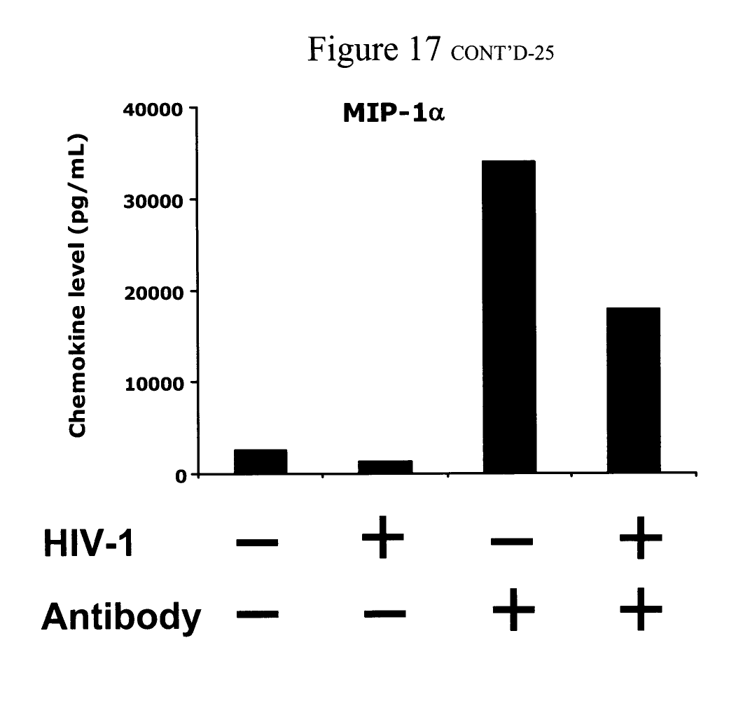

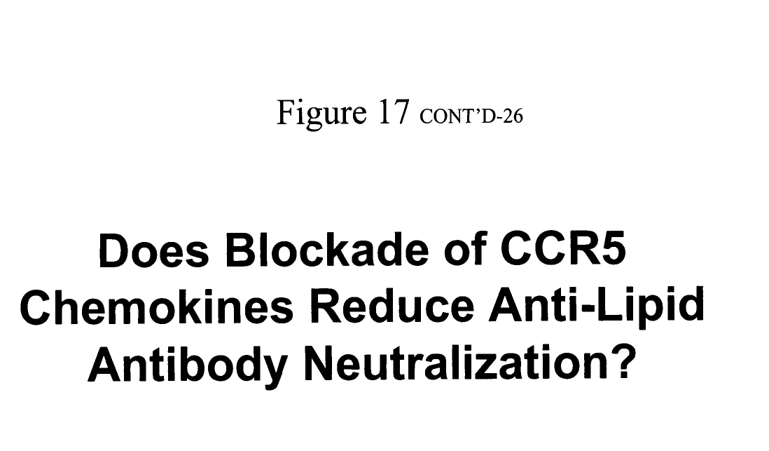

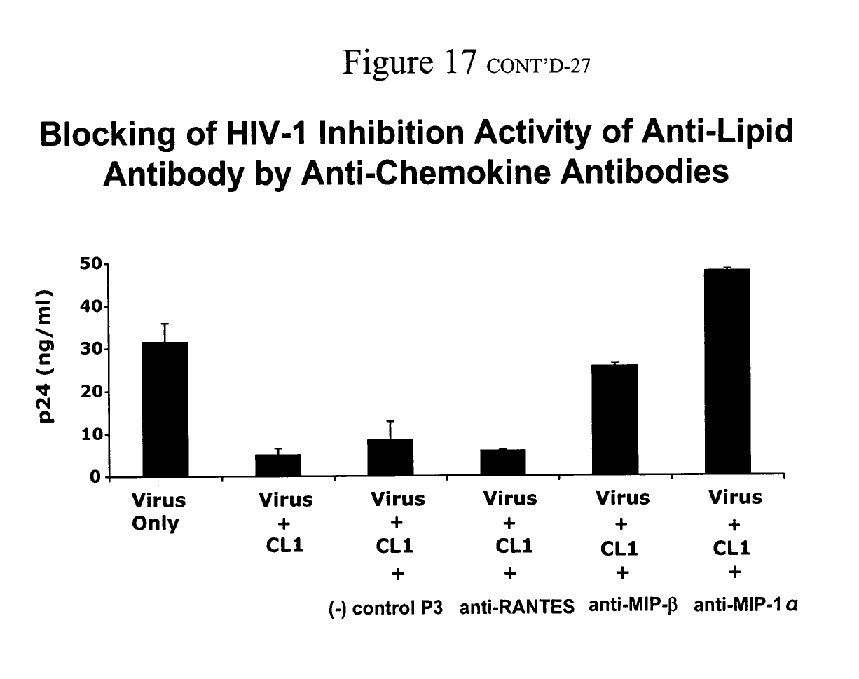

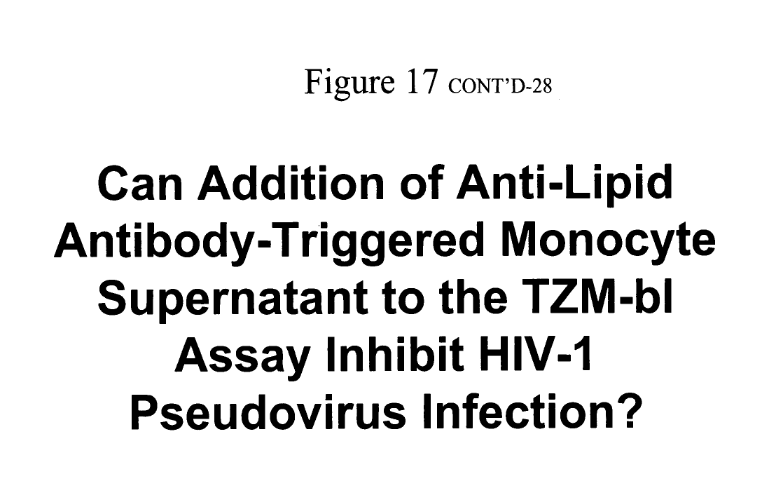

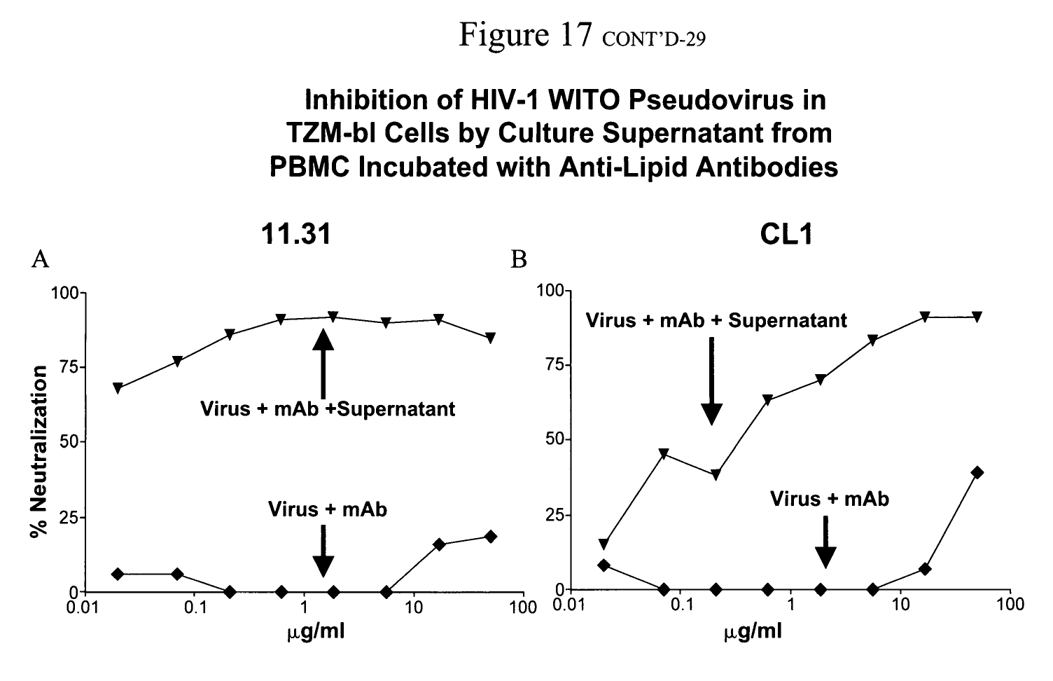

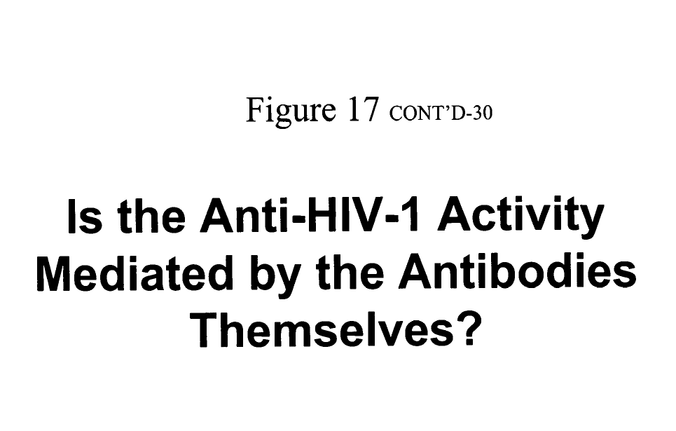

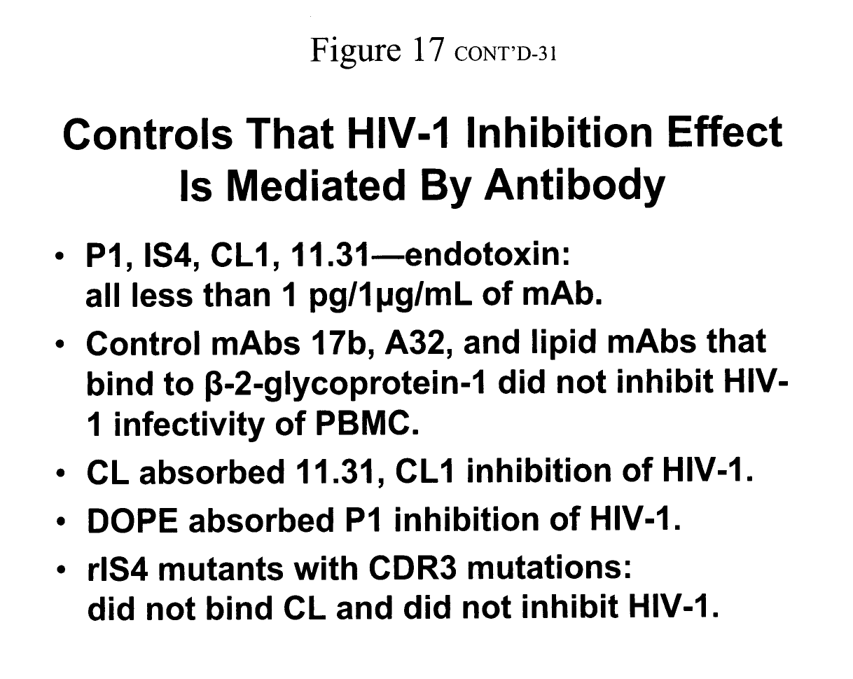

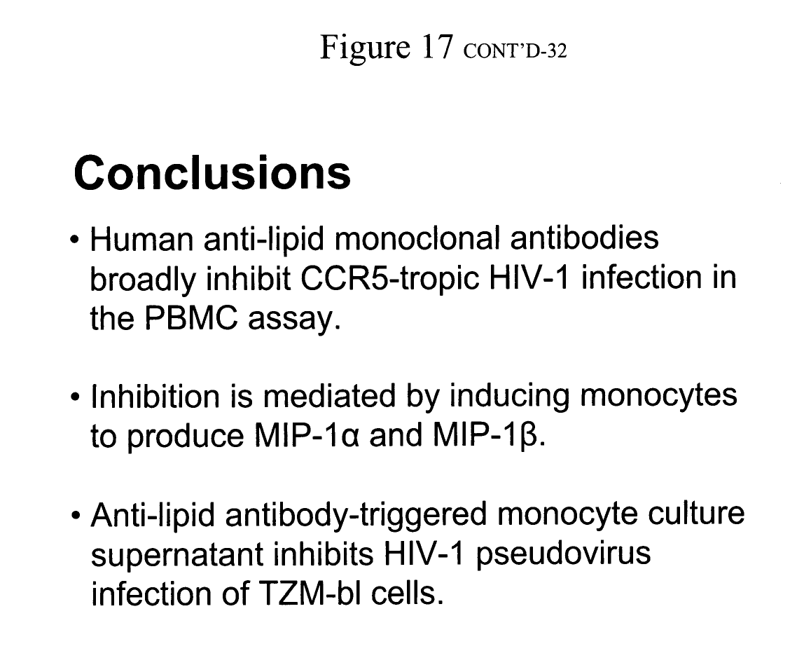

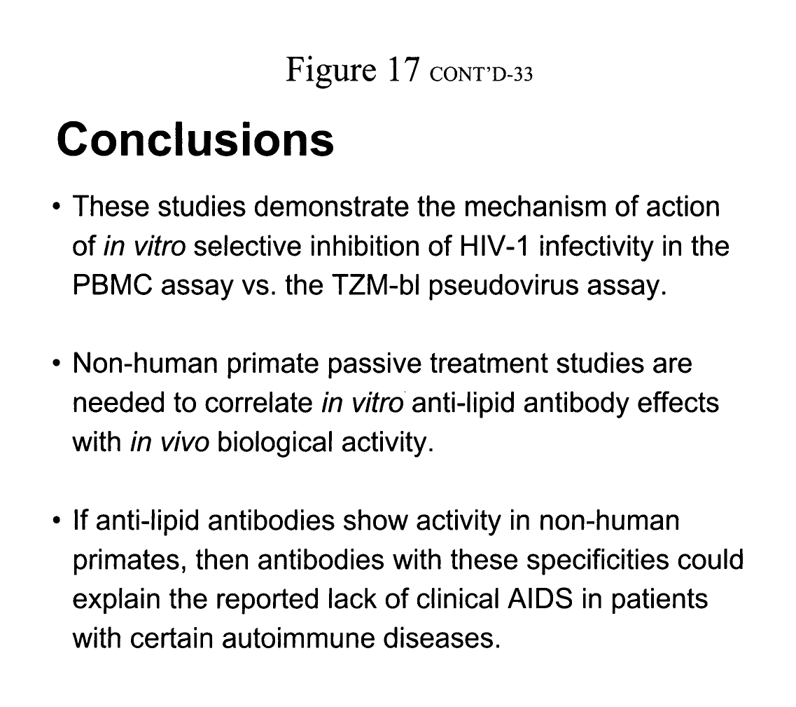

PBMC were incubated with serial dilutions of antibodies 11.31 (FIG. 16A) or CL1 (FIG. 16B) for 24 hours in the presence of HIV-1 WITO transmitted envelope pseudovirus and then the supernatants with antibody and pseudovirus were added to TZM-bl cell cultures. Data shown are inhibition of infection compared to a control sample. For both monoclonal antibodies, inhibition of infection occurred when PBMC-conditioned supernatants were added but was not seen when antibody alone was added. Inhibition curves were similar for PBMC incubated with antibodies alone or for the standard PBMC assay (data not shown). (See Also FIG. 17.)

All documents and other information sources cited above are hereby incorporated in their entirety by reference.

What is claimed is:

1. A method of inhibiting infection of susceptible cells of a human subject by a CCR5-tropic strain of HIV-1 comprising administering to said subject monoclonal antibody CL1, or fragment thereof, in an amount and under conditions such that said antibody, or said fragment thereof, binds to cells of said subject that:
i) produce CCR5-binding chemokines, and
ii) have on their cell surface an antigen recognized by said antibody, or said fragment thereof, so that production of said chemokines by said cells is induced, either by said antibody, or said fragment thereof, alone or in the presence of said strain of HIV-1, to a level sufficient to inhibit infection of said susceptible cells,
wherein said antibody, or said fragment thereof, is administered within 48 hours of exposure of said human subject to said strain of HIV-1.

2. The method according to claim 1 wherein said susceptible cells are T cells.

3. The method according to claim 1 wherein said fragment is a scFv, Fv, Fab′, Fab or F (ab′)₂ fragment.

4. The method according to claim 1 wherein said antibody, or said fragment thereof, is administered topically.

5. The method according to claim 4 wherein said antibody, or said fragment thereof, is administered to a mucosal surface of said subject.

6. A method of inhibiting infection of susceptible cells of a human subject by a CCR5-tropic strain of HIV-1 comprising administering to said subject an antibody having the binding specificity of CL1, or fragment thereof, in an amount and under conditions such that said antibody, or said fragment thereof, binds to cells of said subject that:
i) produce CCR5-binding chemokines, and
ii) have on their cell surface an antigen recognized by said antibody, or said fragment thereof, so that production of said chemokines by said cells is induced, either by said antibody, or said fragment thereof, alone or in the presence of said strain of HIV-1, to a level sufficient to inhibit infection of said susceptible cells,
wherein said antibody, or said fragment thereof, is administered within 48 hours of exposure of said human subject to said strain of HIV-1.

7. The method according to claim 6 wherein said susceptible cells are T cells.

8. The method according to claim 6 wherein said fragment is a scFv, Fv, Fab′, Fab or F (ab′)₂ fragment.

9. The method according to claim 6 wherein said antibody, or said fragment thereof, is administered topically.

10. The method according to claim 6 wherein said antibody, or said fragment thereof, is administered to a mucosal surface of said subject.


SEQUENCE LISTING (10 sequences)

SEQ ID NO: 1 - DNA, 378 bp, Artificial Sequence
  Description of Artificial Sequence Synthetic polynucleotide
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgaggagtcgttttaggtcgtggctggtaaagcgccggtatgtctactttgactactggggccagggaaccctggtcaccgtctcctca

SEQ ID NO: 2 - DNA, 321 bp, Artificial Sequence
  Description of Artificial Sequence Synthetic polynucleotide
tcctctgagctgagtcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaacgtggtattcggcggagggaccaaggtgaccgtccta

SEQ ID NO: 3 - protein, 126 aa, Artificial Sequence
  Description of Artificial Sequence Synthetic polypeptide
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARSRFRSWLVKRRYVYFDYWGQGTLVTVSS

SEQ ID NO: 4 - protein, 107 aa, Artificial Sequence
  Description of Artificial Sequence Synthetic polypeptide
SSELSQDPAVSVALGQTVRITCQGDSLRSYYASWYQQKPGQAPVLVIYGKNNRPSGIPDRFSGSSSGNTASLTITGAQAEDEADYYCNSRDSSGNVVFGGGTKVTVL

SEQ ID NO: 5 - protein, 456 aa, Artificial Sequence
  Description of Artificial Sequence Synthetic polypeptide
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARSRFRSWLVKRRYVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 213 aa, Artificial Sequence
  Description of Artificial Sequence Synthetic polypeptide
SSELSQDPAVSVALGQTVRITCQGDSLRSYYASWYQQKPGQAPVLVIYGKNNRPSGIPDRFSGSSSGNTASLTITGAQAEDEADYYCNSRDSSGNVVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - DNA, 1655 bp, Artificial Sequence
  Description of Artificial Sequence Synthetic polynucleotide
aagcctggctagcacaactcctcaccatggactggacctggacgatcctcttcttgctggcagcagccacaggagcccactcccgggtgcagctggtgcagtctggggctgaggtgaaaaggcctggggcctcggtgaggctctcctgcaaggcttcgggatactccttcactgactactatctccactgggtccgtcaggcccctggacaaggcctcgagtggctggggcgggtcagccctaaaagtggtggcacaagccttctgtcgaagtttcagggcagggtcgccatgaccagtgacacgtccgtcactacagcctacatggaactgaccagcctgacatctgacgacacggccgtctatttctgtgcgagaggcgatgtgaaccggaactggctcgacccctggggcccgggaaccctggtcaccgtctcctcaccttccaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggataagagagttgagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccatgcccacggtgcccagcacctgaactcctgggaggaccgtcagtcttcctcttccccccaaaacccaaggatacccttatgatttcccggacccctgaggtcacgtgcgtggtggtgtacgtgagccacgaagaccccgaggtccagttcaagtggtacgtggacggcgtggaggtgcataatgccaagacaaagccgcgggaggagcagttcaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgagtgcgacggccggcaagcccccgctccccgggctctcggggtcgcgcgaggatgcttggcacgtaccccgtgtacatactt

SEQ ID NO: 8 - protein, 515 aa, Artificial Sequence
  Description of Artificial Sequence Synthetic polypeptide
MDWTWTILFLLAAATGAHSRVQLVQSGAEVKRPGASVRLSCKASGYSFTDYYLHWVRQAPGQGLEWLGRVSPKSGGTSLLSKFQGRVAMTSDTSVTTAYMELTSLTSDDTAVYFCARGDVNRNWLDPWGPGTLVTVSSPSTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVYVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 9 - DNA, 778 bp, Artificial Sequence
  Description of Artificial Sequence Synthetic polynucleotide
gcctcaggaagcagcatcggggggtgccgcagccatggcctggaccgctctccttctgagcctccttgctcactttacagcttctgtggcctcctatgaactgactcagccaccctcagtgtcactggccctggggcagacggccacaattacctgtggcggaaacaacattggctataagaatgtgcactggtaccagcagcagccaggccaggcccctgtgctggtcatctatagggatagcaaccggccccctgggatccctgaccggttctctggctccaactcgcggcacaaggccaccctgaccatcagcagagcccaagccggggatgaggctgactatttctgtcaggtgtgggacagcagcactggatttgttgttttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataggttctcaaccctcacccccccaccacgggagactagagctgc

SEQ ID NO: 10 - protein, 233 aa, Artificial Sequence
  Description of Artificial Sequence Synthetic polypeptide
MAWTALLLSLLAHFTASVASYELTQPPSVSLALGQTATITCGGNNIGYKNVHWYQQQPGQAPVLVIYRDSNRPPGIPDRFSGSNSRHKATLTISRAQAGDEADYFCQVWDSSTGFVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS